(19) United States
(12) Patent Application Publication
(10) Pub. No.: US 2005/0171020 A1
(43) Pub. Date: Aug. 4, 2005

(54) Growth factor polypeptides and nucleic acids encoding same

(76) Inventors: Richard A. Shimkets, West Haven, CT (US); Henri Lichenstein, Madison, CT (US); John L. Herrmann, Guilford, CT (US); Ferenc L. Boldog, North Haven, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/096,308

(22) Filed: Apr. 1, 2005

Related U.S. Application Data
(63) Continuation of application No. 09/662,783, filed on Sep. 12, 2000.
(60) Provisional application No. 60/158,083, filed on Oct. 7, 1999.
(60) Provisional application No. 60/186,707, filed on Mar. 3, 2000.
(60) Provisional application No. 60/188,250, filed on Mar. 10, 2000.
(60) Provisional application No. 60/159,231, filed on Oct. 13, 1999.
(60) Provisional application No. 60/174,485, filed on Jan. 4, 2000.
(60) Provisional application No. 60/223,879, filed on Aug. 8, 2000.

(51) Int. Cl. A61K 38/18

(52) U.S. Cl. 514/12

(57) ABSTRACT

Disclosed are novel nucleic acids encoding proteins and polypeptides related to bone morphogenetic protein-1 (BMF1) and to vascular endothelial growth factor E (VEGF-E).

### RELATED APPLICATIONS

This application is a continuation of U.S. Ser. No. 09/662,783, filed Sep. 12, 2000, which claims priority to U.S. Ser. No. 60/158,083, filed Oct. 7, 1999, U.S. Ser. No. 60/186,707 filed Mar. 3, 2000, U.S. Ser. No. 60/188,250, filed Mar. 10, 2000, U.S. Ser. No. 60/159,231, filed Oct. 13, 1999, U.S. Ser. No. 60/174,485 filed Jan. 4, 2000 and U.S. Ser. No. 60/223,879, Aug. 8, 2000. Each of these applications is incorporated herein by reference in its entirety.

### FIELD OF THE INVENTION

The invention relates to nucleic acids and polypeptides. In particular, this invention discloses novel nucleic acids and polypeptides with growth factor activity in mammals. Additionally antibodies specific for the polypeptides are disclosed.

### BACKGROUND OF THE INVENTION

Polypeptide growth factors exerting effects in a variety of tissues have been described. Among these growth factors are bone morphogenetic protein-1 (BMP-1), vascular endothelial growth factor (VEGF), and platelet-derived growth factor (PDGF).

Multiple effects have been attributed to BMP-1. For example, BMP-1 is capable of inducing formation of cartilage in vivo. BMP1 is also identical to purified procollagen C proteinase (PCP), a secreted calcium-dependent metalloprotease that has been reported to be required for cartilage and bone formation, BMP-1 cleaves the C-terminal propeptides of procollagen I, II, and III and its activity is increased by the procollagen C-endopeptidase enhancer protein.

Vascular endothelial growth factor (VEGF) polypeptides have been reported to act as mitogens primarily for vascular endothelial cells. The specificity for vascular endothelial cells contrasts VEGF polypeptides from other polypeptide mitogens, such as basic fibroblast growth factor and platelet-derived growth factors, which are active on a wider range of cell types.

VEGF has also been reported to affect tumor angiogenesis. For example, VEGF has been shown to stimulate the elongation, network formation, and branching of nonproliferating endothelial cells in culture that are deprived of oxygen and nutrients.

Platelet derived growth factor (PDGF) occurs as an A chain and a B chain, which dimerize in solution to form homodimers AA and BB, as well as the heterodimer, AB.

Expression of RNA encoding the A and B subunits of PDGF has been reported in vascular tissues involved in atherosclerosis. PDGF A and B chain mRNA have been reported to be present in mesenchymal-appearing intimal cells and endothelial cells, respectively, of atherosclerotic plaques. In addition, PDGF receptor mRNA has also been localized predominantly in plaque intimal cells.

The B chain of PDGF is related to the transforming gene (v-sis) of simian sarcoma virus. The B chain has also been reported to be mitogen for cells of mesenchymal origin. The B chain has in addition been implicated in autocrine growth stimulation in the pathologic proliferation of endothelial cells characteristically found in glioblastomas. PDGF has also been reported to promote cellular proliferation and inhibits apoptosis.

### SUMMARY OF THE INVENTION

The invention is based in part on the discovery of nucleic acids encoding polypeptides related to bone-morphogenetic protein-1 (BMP-1), vascular endothelial growth factor (VEGF-E) and platelet derived growth factor (PDGF). Included in the invention are FCTR1 nucleic acids and polypeptides including at least SEQ ID NOS: 1 and 2, and FCTR2 nucleic acids and polypeptides including at least SEQ ID NOS:3 and 4. Unless indicated otherwise, these are referred to herein as “FCTRX” nucleic acids and polypeptides.

In one aspect, the invention provides an isolated FCTRX polypeptide or fragment of a FCTRX polypeptide. The FCTRX polypeptide can include, e.g., an amino acid sequence selected from the group consisting of SEQ ID NO:2 and SEQ ID NO:4. Also within the invention is a FCTRX polypeptide that includes the amino acid sequence of a variant of a SEQ ID NO:2 or SEQ ID NO:4 amino acid sequences. In some embodiments, one or more of the amino acids in the variant sequence is changed to a different amino acid. In some embodiments, no more than 15% of the amino acid residues in the amino acid sequence of said variant are changed. A FCTRX polypeptide of the invention also includes a mature form of a SEQ ID NO:2 or SEQ ID NO:4 polypeptide, e.g., a polypeptide having the amino acid sequence of amino acids 24-370 of SEQ ID NO:2, or the corresponding fragments in SEQ ID NO:4. In other embodiments, the invention includes a variant of a mature form of a polypeptide including amino acid sequence of SEQ ID NO:2 and SEQ ID NO:4. In the variant form, one or more of the amino acids specified in the chosen sequence is changed to a different amino acid. In some embodiments, no more than 15% of the amino acid residues in the amino acid sequence of the variant of said mature form differ from the sequence of a SEQ ID NO:2 or SEQ ID NO:4 polypeptide.

Also provided by the invention is a fragment of a FCTRX polypeptide, a fragment of a variant form of a FCTRX polypeptide, a fragment of a mature form of a FCTRX polypeptide, or the fragment of a variant of a mature form a FCTRX polypeptide. Fragments of a FCTRX polypeptide include, e.g., amino acids 247-370 of SEQ ID NO:2, amino acids 247-338 of SEQ ID NO:2, and amino acids 339-370 of SEQ ID NO:2, as well as the corresponding homologous fragments in SEQ ID NO:4.

The invention also provides FCTRX nucleic acid molecules, including nucleic acid molecules, such as SEQ ID NOs:1 and 3, encoding FCTRX polypeptides, nucleic acids encoding variants of FCTRX polypeptides, nucleic acids encoding mature forms of FCTRX polypeptides, or nucleic acids encoding variants of mature forms of FCTRX polypeptides.

The invention also features an antibody that immunoselectively-binds to FCTRX polypeptides. The antibody can be, e.g., a monoclonal antibody, a humanized antibody, or a human antibody.

In another aspect, the invention includes pharmaceutical compositions that include therapeutically- or prophylactically-effective amounts of a therapeutic and a pharmaceutically-acceptable carrier. The therapeutic can be, e.g., a FCTRX nucleic acid, a FCTRX polypeptide, or an antibody specific for a FCTRX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a FCTRX nucleic acid under conditions allowing for expression of the FCTRX polypeptide encoded by the FCTRX nucleic acid. If desired, the FCTRX polypeptide can then be recovered.

In another aspect, the invention includes a method of detecting the presence of a FCTRX polypeptide in a sample. In the method, a sample is contacted with a compound that selectively binds to the polypeptide under conditions allowing for formation of a complex between the polypeptide and the compound. The complex is detected, if present, thereby identifying the FCTRX polypeptide within the sample. The compound can be, e.g., an ant-FCTRX antibody, or another polypeptide that binds to a FCTRX polypeptide.

Also included in the invention is a method of detecting the presence of a FCTRX nucleic acid molecule in a sample by contacting the sample with a FCTRX nucleic acid probe or primer, and detecting whether the nucleic acid probe or primer bound to a FCTRX nucleic acid molecule in the sample.

In a further aspect, the invention provides a method for modulating the activity of a FCTRX polypeptide. The method includes contacting a cell sample that includes the FCTRX polypeptide with a compound that binds to the FCTRX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

The invention further includes a method for screening for a modulator of disorders or syndromes including, e.g., cancer. The method includes contacting a test compound with a FCTRX polypeptide and determining if the test compound binds to said FCTRX polypeptide. Binding of the test compound to the FCTRX polypeptide indicates the test compound is a modulator of activity, or of latency or predisposition to the disorder or syndrome. In one embodiment, the candidate test compound has a molecular weight not more than about 1500 Da.

Also within the scope of the invention is a method for screening for a modulator of activity, or of latency or predisposition to an disorders or syndromes including, e.g., anxiety disorders; CNS disorders, diabetes, obesity; and infectious disease, by administering a test compound to a test animal at increased risk for the aforementioned disorders or syndromes. The test animal expresses a recombinant polypeptide encoded by a FCTRX nucleic acid. Expression or activity of FCTRX polypeptide is then measured in the test animal, as is expression or activity of the protein in a control animal which recombinantly-expresses FCTRX polypeptide and is not at increased risk for the disorder or syndrome. Next, the expression of FCTRX polypeptide in both the test animal and the control animal is compared. A change in the activity of FCTRX polypeptide in the test animal relative to the control animal indicates the test compound is a modulator of latency of the disorder or syndrome.

In yet another aspect, the invention includes a method for determining the presence of or predisposition to a disease associated with altered levels of a FCTRX polypeptide, a FCTRX nucleic acid, or both, in a subject (e.g., a human subject). The method includes measuring the amount of the FCTRX polypeptide in a test sample from the subject and comparing the amount of the polypeptide in the test sample to the amount of the FCTRX polypeptide present in a control sample. An alteration in the level of the FCTRX polypeptide in the test sample as compared to the control sample indicates the presence of or predisposition to a disease in the subject. Preferably, the predisposition includes, e.g., cancers, cell proliferation disorders, anxiety disorders; CNS disorders; diabetes; obesity; and infectious disease.

In a further aspect, the invention includes a method of treating or preventing a pathological condition associated with a disorder in a mammal by administering to the subject a FCTRX polypeptide, a FCTRX nucleic acid, or a FCTRX-specific antibody to a subject (e.g., a human subject), in an amount sufficient to alleviate or prevent the pathological condition. In preferred embodiments, the disorder, includes, e.g., cancers, cell proliferation disorders, anxiety disorders, CNS disorders, diabetes; obesity; and infectious disease.

FCTRX nucleic acids according to the invention can be used to identify various cell types, including cancerous cells. For example, Example 7 illustrates that clone 30664188.0.99 (SEQ ID NO:1) is strongly expressed specifically in CNS cancer, lung cancer and ovarian cancer. It is also shown in the Examples that SEQ ID NO:1 produces a gene product which either persists intact in conditioned medium arising from transfecting HEK 293 cells, or is proteolytically cleaved. Evidence presented in Example 13 suggests that the form of the 30664188.0.99 protein (SEQ ID NO:2) that is active in the various experiments, which are reported in the Examples, is a proteolysis product of the 30664188.0.99 protein. As shown in the Examples, the activities ascribed to either one or both of these substances include the ability to stimulate net DNA synthesis as monitored by incorporation of BrdU into DNA, proliferation of cell number, the ability to transform cells in culture, and the ability to induce tumor formation in vivo. These various activities occur in a variety of cell types.

FCTRX nucleic acids, and their encoded polypeptides, can also be used to modulate cell growth. For example, it is likely that the polypeptide having the amino acid sequence of SEQ ID NO:2 or SEQ ID NO:4, or both, has specific functions in a variety of cells. In addition to stimulating growth and proliferation of certain cells, it is endogenously expressed in certain specific classes of tumor cell lines. Thus, a FCTRX polypeptide, e.g., a polypeptide having the amino acid sequence of SEQ ID NO:2 or SEQ ID NO:4, can be used where net cell growth and proliferation is desired and in different circumstances where cell growth is to be inhibited or abrogated.

A FCTRX nucleic acid or gene product, e.g., a nucleic acid encoding SEQ ID NO:2 or SEQ ID NO:4, is useful as a therapeutic agent in promoting wound healing, neovascularization and tissue growth, and similar tissue regeneration needs. More specifically, a FCTRX nucleic acid or polypeptide may be useful in treatment of anemia and leukopenia, intestinal tract sensitivity and baldness. Treatment of such conditions may be indicated in, e.g., patients having undergone radiation or chemotherapy. It is intended in such cases that administration of a FCTRX nucleic acid or polypeptide, e.g., a polypeptide including the amino acid sequence of SEQ ID NO:2 or SEQ ID NO:4, or a nucleic acid sequence encoding these polypeptides (e.g., SEQ ID NO:1 or SEQ ID NO:3) will be controlled in dose such that any hyperproliferative side effects are minimized.

Alternatively, in cases of tumors, such as CNS cancer and ovarian cancer, in which FCTRX nucleic acids is expressed at high levels, (e.g., a tumor in SEQ ID NO:1 is expressed in high levels), it is desired to inhibit or eliminate the effects of production of a FCTRX nucleic acid or gene product (e.g., SEQ ID NO:2 or SEQ ID NO:4, or a nucleic acid encoding one of these polypeptides). For example, this may be accomplished by administration of an antibody directed against a polypeptide having the amino acid sequence of SEQ ID NO:2 or SEQ ID NO:4, or fragment thereof. In particular, the antibody can be directed against the active fragment p35 (see the Examples) identified herein. An alternative example involves identifying the putative protease implicated in the formation of p35 from p85 (see the Examples). Administration of a substance that specifically inhibits the activity of this protease, but not the activity of other proteases, will be effective to prevent formation of the active p35 form of a FCTRX polypeptide, e.g., a clone 30664188.0.99 polypeptide.

Based on the roles of molecules related to FCTRX polypeptides and nucleic acids, (e.g., BMP-1, VEGF-like polypeptides such as fallotein, and PDGF) in malignant disease progression and the gene expression profile described herein, it is foreseen that, for a subset of human gliomas and ovarian epithelial carcinomas, targeting of a FCTRX polypeptide using an antibody has an inhibitory effect on tumor growth, matrix invasion, chemo-resistance, radio-resistance, and metastatic dissemination. In various embodiments, the FCTRX polypeptide is linked to a monoclonal antibody, a humanized antibody or a fully human antibody.

A FCTRX polypeptide can potentially block or limit the extent of tumor neovascularization. In addition to classical modes of administration of potential antibody therapeutics newly developed modalities of administration may be useful. For example, local administration of ¹³¹I-labeled monoclonal antibody for treatment of primary brain tumors after surgical resection has been reported. Additionally, direct stereotactic intracerebral injection of monoclonal antibodies and their fragments is also being studied clinically and pre-clinically. Intracarotid hyperosmolar perfusion is an experimental strategy to target primary brain malignancy with drug conjugated human monoclonal antibodies.

Additionally, the nucleic acids of the invention, and fragments and variants thereof, may be used, by way of nonlimiting example, (a) to direct the biosynthesis of the corresponding encoded proteins, polypeptides, fragments and variants as recombinant or heterologous gene products, (b) as probes for detection and quantification of the nucleic acids disclosed herein, (c) as sequence templates for preparing antisense molecules, and the like. Such uses are described more fully in the following disclosure.

Furthermore, the proteins and polypeptides of the invention, and fragments and variants thereof, may be used, in ways that include (a) serving as an immunogen to stimulate the production of an anti-FCTRX antibody, (b) a capture antigen in an immunogenic assay for such an antibody, and (c) as a target for screening for substances that bind to a FCTRX polypeptide of the invention. These utilities and other utilities for FCTRX nucleic acids, polypeptides, antibodies, agonists, antagonists, and other related compounds uses are disclosed more fully below.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE FIGURES

FIG. 1 is a representation of an alignment of the amino acid sequence of clone 30664188.0.99 with the amino acid sequence of a human secretory growth factor-like protein VEGF-E amino acid sequence (SEQ ID NO:24).

FIG. 2 is a representation of a Western blot of a 30664188.m99 protein expressed in E. coli cells.

FIG. 3 is a representation of a Western blot of a 30664188.m99 protein secreted by human 293 cells.

FIG. 4A is a schematic representation of a scheme for the recombinant production, purification and apparent molecular weight of a mature form of the protein of clone 30664188.0.99.

FIG. 4B includes representations of two Western blot analyses showing expression of a 30664188.0.m99 polypeptide.

FIG. 5 is a graph showing incorporation of BrdU into NIH 3T3 cells and CCD-1070 cells in response to various treatments.

FIG. 6 is a graph showing proliferation of NIH 3T3 5-24 cells in response to various treatments.

FIG. 7 is a graph showing cell number in NIH 3T3 cells exposed to a mock treatment or 30664188.

FIG. 8 is a depiction of a photomicrograph showing cell density and cell morphology of NIH 3T3 cells in response to treatment with pCEP4sec CM or 30664188 protein.

FIG. 9 is a depiction of a photomicrograph showing changes in cell number in NHost osteoblast cells in response to various treatments.

FIG. 10A is a representation of a western blot of 30664188.m99 expressed by HEK 293 cells cultured in the absence of serum.

FIG. 10B is a representation of SDS-PAGE 30664188.m99 protein expressed by HEK 293 cells cultured in the presence of serum.

FIG. 11 is a representation of dose titration of BrdU incorporation into NIH 3T3 cells stimulated by p85 and by p35 fragments of 30664188.m99 protein.

### DETAILED DESCRIPTION OF THE INVENTION

The invention provides nucleic acids that encoded polypeptides related to bone-morphogen protein-1 (BMP-1) and vascular endothelial growth factor (VEGF-E). Nucleic acids and polypeptides of the invention include FCTR1 nucleic acids and polypeptides (e.g., the nucleic acid sequence disclosed in SEQ ID NO:1 and SEQ ID NO:2, respectively). The invention also provides FCTR2 nucleic acids and polypeptides. Examples of FCTR2 nucleic acids and polypeptides include the nucleic acid sequence and polypeptide sequence disclosed in SEQ ID NO:3 and SEQ ID NO:4, respectively. Unless otherwise indicated, the FCTR1 and FCTR2 nucleic acids and polypeptides, and compositions and methods based on these nucleic acids and polypeptides, are referred to as FCTRX nucleic acids or polypeptides. FCTR1 Nucleic Acids and Polypeptides A FCTR1 polynucleotide of the invention includes the nucleic acid present in clone 30664188.0.99. Clone 30664188.0.99 is 1828 nucleotides in length. The nucleotide sequence of FCTR1 (also referred to as 30664188.0.99) is reported in Table 1 (SEQ ID NO:1). The clone was originally obtained from RNA from pituitary gland tissues is also present in RNA from human uterine microvascular endothelial cells (Clonetics, San Diego, Calif.), human erythroleukemia cells (ATCC, Manassas, Va.), thyroid, small intestine, lymphocytes, adrenal gland and salivary gland.

TABLE 1

NUCLEOTIDE (SEQ ID NO:1) AND PROTEIN (SEQ
ID NO:2) SEQUENCE OF FCTR1 (also referred
to as 30664188-0-99 Translated Protein-
Frame: 2-Nucleotide 182 to 1291

| | |
|---|---|
| 1 | CTAAAAAATATGTTCTCTACAACACCAAGGCTCATTAAAATATTT |
| 46 | TAAATATTAATATACATTTCTTCTGTCAGAAATACATAAAACTTT |
| 91 | ATTATATCAGCGCAGGGCGGCGCGGCGTCGGTCCCGGGAGCAGAA |
| 136 | CCCGGCTTTTTCTTGGAGCGACGCTGTCTCTAGTCGCTGATCCCA |
| 181 | AATGCACCGGCTCATCTTTGTCTACACTCTAATCTGCGCAAACTT |
| | MetHisArgLeuIlePheValTyrThrLeuIleCysAlaAsnPh |
| 226 | TTGCAGCTGTCGGGACACTTCTGCAACCCCGCAGAGCGCATCCAT |
| | eCysSerCysArgAspThrSerAlaThrProGlnSerAlaSerIl |
| 271 | CAAAGCTTTGCGCAACGCCAACCTCAGGCGAGATGAGAGCAATCA |
| | eLysAlaLeuArgAsnAlaAsnLeuArgArgAspGluSerAsnHi |
| 316 | CCTCACAGACTTGTACCGAAGAGATGAGACCATCCAGGTGAAAGG |
| | sLeuThrAspLeuTyrArgArgAspGluThrIleGlnValLysGl |
| 361 | AAACGGCTACGTGCAGAGTCCTAGATTCCCGAACAGCTACCCCAG |
| | yAsnGlyTyrValGlnSerProArgPheProAsnSerTyrProAr |
| 406 | GAACCTGCTCCTGACATGGCGGCTTCACTCTCAGGAGAATACACG |
| | gAsnLeuLeuLeuThrTrpArgLeuHisSerGlnGluAsnThrAr |
| 451 | GATACAGCTAGTGTTTGACAATCAGTTTGGATTAGAGGAAGCAGA |
| | gIleGlnLeuValPheAspAsnGlnPheGlyLeuGluGluAlaGl |
| 496 | AAATGATATCTGTAGGTATGATTTTGTGGAAGTTGAAGATATATC |
| | uAsnAspIleCysArgTyrAspPheValGluValGluAspIleSe |
| 541 | CGAAACCAGTACCATTATTAGAGGACGATGGTGTGGACACAAGGA |
| | rGluThrSerThrIleIleArgGlyArgTrpCysGlyHisLysGl |
| 586 | AGTTCCTCCAAGGATAAAATCAAGAACGAACCAAATTAAAATCAC |
| | uValProProArgIleLysSerArgThrAsnGlnIleLysIleTh |
| 631 | ATTCAAGTCCGATGACTACTTTGTGGCTAAACCTGGATTCAAGAT |
| | rPheLysSerAspAspTyrPheValAlaLysProGlyPheLysIl |
| 676 | TTATTATTCTTTGCTGGAAGATTTCCAACCCGCAGCAGCTTCAGA |
| | eTyrTyrSerLeuLeuGluAspPheGlnProAlaAlaAlaSerGl |
| 721 | GACCAACTGGGAATCTGTCACAAGCTCTATTTCAGGGGTATCCTA |
| | uThrAsnTrpGluSerValThrSerSerIleSerGlyValSerTy |
| 766 | TAACTCTCCATCAGTAACGGATCCCACTCTGATTGCGGATGCTCT |
| | rAsnSerProSerValThrAspProThrLeuIleAlaAspAlaLe |
| 811 | GGACAAAAAAATTGCAGAATTTGATACAGTGGAAGATCTGCTCAA |
| | uAspLysLysIleAlaGluPheAspThrValGluAspLeuLeuLy |
| 856 | GTACTTCAATCCAGAGTCATGGCAAGAAGATCTTGAGAATATGTA |
| | sTyrPheAsnProGluSerTrpGlnGluAspLeuGluAsnMetTy |
| 901 | TCTGGACACCCCTCGGTATCGAGGCAGGTCATACCATGACCGGAA |
| | rLeuAspThrProArgTyrArgGlyArgSerTyrHisAspArgLy |
| 946 | GTCAAAAGTTGACCTGGATAGGCTCAATGATGATGCCAAGCGTTA |
| | sSerLysValAspLeuAspArgLeuAsnAspAspAlaLysArgTy |
| 991 | CAGTTGCACTCCCAGGAATTACTCGGTCAATATAAGAGAAGAGCT |
| | rSerCysThrProArgAsnTyrSerValAsnIleArgGluGluLe |
| 1036 | GAAGTTGGCCAATGTGGTCTTCTTTCCACGTTGCCTCCTCGTGCA |
| | uLysLeuAlaAsnValValPhePheProArgCysLeuLeuValGl |
| 1081 | GCGCTGTGGAGGAAATTGTGGCTGTGGAACTGTCAACTGGAGGTC |
| | nArgCysGlyGlyAsnCysGlyCysGlyThrValAsnTrpArgSe |
| 1126 | CTGCACATGCAATTCAGGGAAAACCGTGAAAAAGTATCATGAGGT |
| | rCysThrCysAsnSerGlyLysThrValLysLysTyrHisGluVa |
| 1171 | ATTACAGTTTGAGCCTGGCCACATCAAGAGGAGGGGTAGAGCTAA |
| | lLeuGlnPheGluProGlyHisIleLysArgArgGlyArgAlaLy |
| 1216 | GACCATGGCTCTAGTTGACATCCAGTTGGATCACCATGAACGATG |
| | sThrMetAlaLeuValAspIleGlnLeuAspHisHisGluArgCy |
| 1261 | TGATTGTATCTGCAGCTCAAGACCACCTCGATAAGAGAATGTGCA |
| | SAspCysIleCysSerSerArgProProArg |
| 1306 | CATCCTTACATTAAGCCTGAAAGAACCTTTAGTTTAAGGAGGGTG |
| 1351 | AGATAAGAGACCCTTTTCCTACCAGCAACCAAACTTACTACTAGC |
| 1396 | CTGCAATGCAATGAACACAAGTGGTTGCTGAGTCTCAGCCTTGCT |
| 1441 | TTGTTAATGCCATGGCAAGTAGAAAGGTATATCATCAACTTCTAT |
| 1486 | ACCTAAGAATATAGGATTGCATTTAATAATAGTGTTTGAGGTTAT |
| 1531 | ATATGCACAAACACACACAGAAATATATTCATGTCTATGTGTATA |
| 1576 | TAGATCAAATGTTTTTTTTGGTATATATAACCAGGTACACCAGAG |
| 1621 | CTTACATATGTTTGAGTTAGACTCTTAAAATCCTTTGCCAAAATA |
| 1666 | AGGGATGGTCAAATATATGAAACATGTCTTTAGAAAATTTAGGAG |
| 1711 | ATAAATTTATTTTTAAATTTTGAAACACAAAACAATTTTGAATCT |
| 1756 | TGCTCTCTTAAAGAAAGCATCTTGTATATTAAAAATCAAAAGATG |
| 1801 | AGGCTTTCTTACATATACATCTTAGTTG |

Nucleotides 182 to 1292 of SEQ ID NO:1 encode a 370 amino acid protein (SEQ ID NO:2) that includes sequences characteristic of secreted proteins. The sequence of the encoded protein, which is also referred to herein as “30664188.0.99 protein” or “30664188.0.99”, is presented in Table 1. The predicted molecular weight of the 30664188.0.99 protein is 42847.8 daltons.

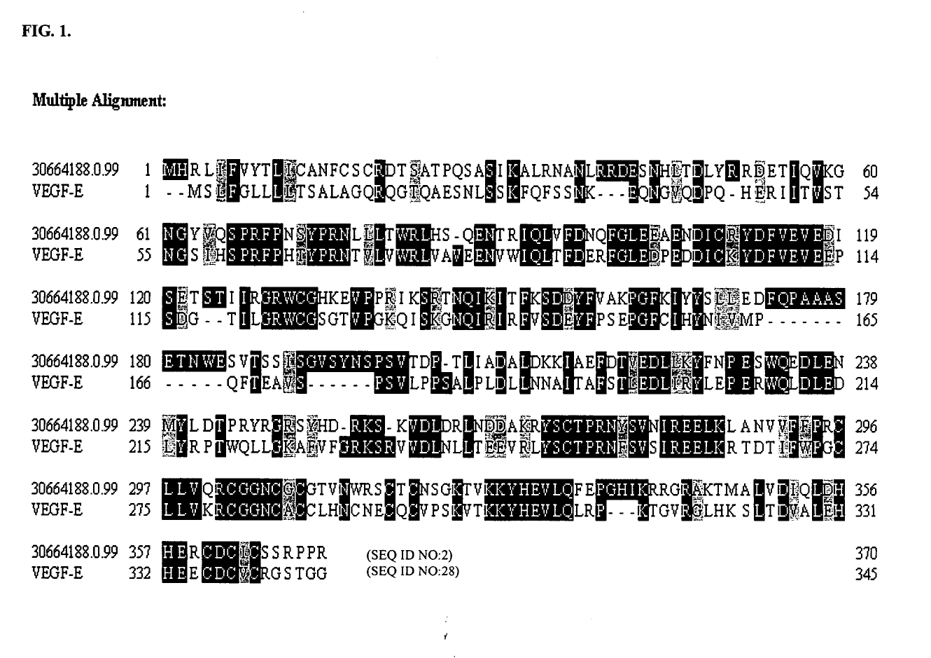

BLASTN and BLASTP analyses indicate the 30664188.0.99 polypeptide has a likeness to human vascular endothelial growth factor E (VEGF-E), as well as to VEGF-E from other vertebrate species. For example, there is a 44% identity to human secretory growth factor-like protein (VEGF-E, or fallotein; Acc. No.: AAF00049 which references GenBank-ID: AF091434 for the nucleotide sequence). An alignment of the amino acid sequence of the 30664188.0.99 polypeptide with that of VEGF-E is shown in FIG. 1.

The 30664188.0.99 polypeptide amino acid sequence shows similarity to the sequence of human procollagen C-endopeptidase (bone morphogenetic protein-1; BMP-1; PIR-ID:A58788), which is a polypeptide of 823 residues. Residues 54 to 169 of the 30664188.0.99 polypeptide show 30-41% identity over three segments of the BMP-1 polypeptide. The 30664188.0.99 polypeptide also shows a similar degree of identity is to BMP-1 from Xenopus laevis (ACC NO:P98070), which is a 707 residue protein. The latter protein may act as a zinc protease in promoting cartilage and bone formation (Wozney et al., Science 242: 1528-34, 1988).

The 30664188.0.99 polypeptide is also related to other growth factors. For example, it shows 42% identity and 59% similarity to chicken spinal cord-derived growth factor (TREMBLNEW-ACC:BAB03265), 42% identity and 59% identity to human secretory growth factor-like protein fallotein (SPTREMBL-ACC:Q9UL22), 42% identity and 39% similarity to human platelet-derived growth factor C (TREMBLNEW-ACC:AAF80597), and 39% identity and 59% similarity to mouse fallotein (SPTREMBL-ACC:Q9QY71).

The homologies discussed above identify the 30664188.0.99 polypeptide as a member of the BMP-1/VEGF-E/PDGF protein family. BMP-1 proteins include an EGF-like domain, three CUB domains, and PDGF/VEGF domains. BMP-1 proteins are also members of the astacin subfamily.

SignalP and PSORT analyses predict that the amino acid sequence for 30664188.0.99 includes a cleavable amino terminal signal peptide with a cleavage site between positions 23 and 24 (TSA-TP). The protein is most likely secreted and localized outside of the cell. The InterPro software program predicts the presence of a CUB domain in 30664188.0.99 from residue 53 to residue 167, a PDGF domain spanning residues 272-306 and 350-362, and a metallothionein domain from residue 302 to residue 365. A FCTR1 polypeptide of the invention includes a polypeptide having one, two, three, or four of these domains, or a combination thereof.

A FCTR1 polypeptide of the invention includes a mature form of a FCTR1 polypeptide that includes amino acids 24-370 of SEQ ID NO:2. These sequences are also encoded in a construct encoded by clone 30664188.0.m99, which is described in more detail below. Also within the invention are nucleic acids encoding FCTRX polypeptide fragments that include amino acid sequences 247-370, 247-338, or 339-370, or their variant forms. In some embodiments, the fragments stimulate proliferation of cells. Also within the invention are the FCTRX polypeptide fragments, or their variants, encoded by these nucleic acids.

FCTR2 Nucleic Acids and Polypeptides

A FCTR2 polynucleotide of the invention includes the nucleic acid sequence present in clone 30664188.0.331. Clone 30664188.0.331 is 1587 nucleotides in length and was originally isolated from RNA from pituitary gland tissues. The nucleotide sequence of FCTR2 (also referred to as 30664188.0.331) is shown in Table 2 (SEQ ID NO:3).

TABLE 2

NUCLEOTIDE (SEQ ID NO:3) AND PROTEIN (SEQ
ID NO:4) SEQUENCE OF FCTR2 (30664188-0-331)
Translated Protein-Frame: 3-Nucleotide
540 to 935

| | |
|---|---|
| 1 | AGAGGCTCTCAAATTAGATCAAGAAATGCCTTTAACAGAAGTGAA |
| 46 | GAGTGAACCTGCTCCTGACATGGCGGCTTCACTCTCAGGAGAATA |
| 91 | CACGGATACAGCTAGTGTTTGACAATCAGTTTGGATTAGAGGAAG |
| 136 | CAGAAAATGATATCTGTAGGTATGATTTTGTGGAAGTTGAAGATA |
| 181 | TATCCGAAACCAGTACCATTATTAGAGGACGATGGTGTGGACACA |
| 226 | AGGAAGTTCCTCCAAGGATAAAATCAAGAACGAACCAAATTAAAA |
| 271 | TCACATTCAAGTCCGATGACTACTTTGTGGCTAAACCTGGATTCA |
| 316 | AGATTTATTATTCTTTGCTGGAAGATTTCCAACCCGCAGCAGCTT |
| 361 | CAGAGACCAACTGGGAATCTGTCACAAGCTCTATTTCAGGGGTAT |
| 406 | CCTATAACTCTCCATCAGTAACGGATCCCACTCTGATTGCGGATG |
| 451 | CTCTGGACAAAAAAATTGCAGAATTTGATACAGTGGAAGATCTGC |
| 496 | TCAAGTACTTCAATCCAGAGTCATGGCAAGAAGATCTTGAGAATA |
| M | |
| 541 | TGTATCTGGACACCCCTCGGTATCGAGGCAGGTCATACCATGACC |
| | etTyrLeuAspThrProArgTyrArgGlyArgSerTyrHisAspA |
| 586 | GGAAGTCAAAAGTTGACCTGGATAGGCTCAATGATGATGCCAAGC |
| | rgLysSerLysValAspLeuAspArgLeuAsnAspAspAlaLysA |
| 631 | GTTACAGTTGCACTCCCAGGAATTACTCGGTCAATATAAGAGAAG |
| | rgTyrSerCysThrProArgAsnTyrSerValAsnIleArgGluG |
| 676 | AGCTGAAGTTGGCCAATGTGGTCTTCTTTCCACGTTGCCTCCTCG |
| | luLeuLysLeuAlaAsnValValPhePheProArgCysLeuLeuV |
| 721 | TGCAGCGCTGTGGAGGAAATTGTGGCTGTGGAACTGTCAACTGGA |
| | alGlnArgCysGlyGlyAsnCysGlyCysGlyThrValAsnTrpA |
| 766 | GGTCCTGCACATGCAATTCAGGGAAAACCGTGAAAAAGTATCATG |
| | rgSerCysThrCysAsnSerGlyLysThrValLysLysTyrHisG |
| 811 | AGGTATTACAGTTTGAGCCTGGCCACATCAAGAGGAGGGGTAGAG |
| | luValLeuGlnPheGluProGlyHisIleLysArgArgGlyArgA |
| 856 | CTAAGACCATGGCTCTAGTTGACATCCAGTTGGATCACCATGAAC |
| | laLysThrMetAlaLeuValAspIleGlnLeuAspHisHisGluA |
| 901 | GATGTGATTGTATCTGCAGCTCAAGACCACCTCGATAAGAGAATG |
| | RgCysAspCysIleCysSerSerArgProProArg |
| 946 | TGCACATCCTTACATTAAGCCTGAAAGAACCTTTAGTTTAAGGAG |
| 991 | GGTGAGATAAGAGACCCTTTTCCTACCAGCAACCAAACTTACTAC |
| 1036 | TAGCCTGCAATGCAATGAACACAAGTGGTTGCTGAGTCTCAGCCT |
| 1081 | TGCTTTGTTAATGCCATGGCAAGTAGAAAGGTATATCATCAACTT |
| 1126 | CTATACCTAAGAATATAGGATTGCATTTAATAATAGTGTTTGAGG |
| 1171 | TTATATATGCACAAACACACACAGAAATATATTCATGTCTATGTG |
| 1216 | TATATAGATCAAATGTTTTTTTTGGTATATATAACCAGGTACACC |
| 1261 | AGAGCTTACATATGTTTGAGTTAGACTCTTAAAATCCTTTGCCAA |
| 1306 | AATAAGGGATGGTCAAATATATGAAACATGTCTTTAGAAAATTTA |
| 1351 | GGAGATAAATTTATTTTTAAATTTTGAAACACAAAACAATTTTGA |
| 1396 | ATCTTGCTCTCTTAAAGAAAGCATCTTGTATATTAAAAATCAAAA |
| 1441 | GATGAGGCTTTCTTACATATACATCTTAGTTGATTATTAAAAAAG |
| 1486 | GAAAAATATGGTTTCCAGAGAAAAGGCCAATACCTAAGCATTTTT |
| 1531 | TCCATGAGAAGCACTGCATACTTACCTATGTGGACTATAATAACC |
| 1576 | TGTCTCCAAAAC |

Clone 30664188.0.331 includes an open reading frame from nucleotides 540 to 936. The open reading frame encodes a polypeptide of 132 amino acids (SEQ ID NO:4). The encoded polypeptide is referred to herein as the “30664188.0.331 protein” or the “30664188.0.331 polypeptide”. The predicted amino acid sequence of the 30664188.0.331 nucleic acid sequence is shown in Table 2 (SEQ ID NO:4).

Nucleotides 50 to 1472 of clone 30664188.0.331 are 100% identical to nucleotides 406-1828 of clone 30664188.0.99. The 132 amino acids of the clone 30664188.0.331 protein are 100% identical to the carboxy-terminal region of the protein sequence of 30664188.0.99. Thus, the nucleic acids of clones 30664188.0.99 and 30664188.0.331 are therefore related as splice variants of a common gene.

The 30664188.0.331 protein shows similarity to human growth factor FIGF (c-fos-induced growth factor; ptnr:SPTREMBL-ACC:043915), a member of the platelet-derived growth factor/vascular endothelial growth factor (PDGF/VEGF) family, and to rat vascular endothelial growth factor D (ptnr:SPTREMBL-ACC:035251).

The similarities of the disclosed FCTR1 and FCTR2 polypeptides to previously described BMP-1 and VEG-E polypeptides indicate a similarity of functions by the FCTRX nucleic acids and polypeptides of the invention. These utilities are described in more detail below.

FCTRX nucleic acids and polypeptides may be use to induce formation of cartilage, as BMP-1 is also capable of inducing formation of cartilage in vivo (Wozney et al., Science 242: 1528-1534 (1988)).

An additional use for the FCTRX nucleic acids and polypeptides is in the modulation of collagen formation. Recombinantly expressed BMP1 and purified procollagen C proteinase (PCP), a secreted metalloprotease requiring calcium and needed for cartilage and bone formation, are, in fact, identical. See, Kessler et al., Science 271: 360-62 (1996). BMP-1 cleaves the C-terminal propeptides of procollagen I, II, and III and its activity is increased by the procollagen C-endopeptidase enhancer protein. FCTRX nucleic acids and polypeptides may play similar roles in collagen modulation pathways.

FCTRX nucleic acids and polypeptides can also be used to stage various cancers. For example, bone metastases can almost universally be correlated to the morbidity and mortality of certain prostate cancers. For example, bone morphogenetic proteins are implicated as having important roles in various cancers. Overexpression of bone morphogenetic protein (BMP)-4 and BMP-2 mRNA has been reported in gastric cancer cell lines of poorly differentiated type. See, Katoh et al., J. Gastroenterol 31(1): 137-9 (1996). This observation may have implications regarding the poor prognosis of patients with diffuse osteoplastic bone metastasis of gastric cancer. Additionally, osteosarcomas producing bone morphogenetic protein (BMP) differed in clinical features from those not producing BMP. See, Yoshikawa et al Cancer 56: 1682-7 (1985) They were characterized radiologically by perpendicular spicules, histologically by osteoblastic type cells, and clinically by an increased serum alkaline phosphatase level, relative resistance to preoperative chemotherapy with Adriamycin (doxorubicin) plus high-dose methotrexate, and a tendency to metastasize to other bones and the lungs.

The relatedness of FCTRX polypeptides to VEGF—reveals uses for FCTRX nucleic acids and polypeptides in modulating angiogenesis. Angiogenesis is a process which contributes to the development of new blood vessels. During angiogenesis, new capillaries sprout from existing vessels. See, Risau FASEB J. 9(10): 926-33 (1995); Risau et al., Ann. Rev. Cell Dev Biol. 11: 73-91 (1995). In adult mammals, new blood vessels are produced through angiogenesis. Pathological states in which angiogenesis contributes to the appearance and maintenance of the pathology include tumor development and growth. vascular endothelial growth factor F has been reported to be involved in angiogenesis.

Vascular endothelial growth factor (VEGF) is a multifunctional cytokine expressed and secreted at high levels by many tumor cells in both nonhumans and humans. See review in Ferrara, Curr Top Microbiol Immunol 237: 1-30 (1999). VEGF exerts its effects on the vascular endothelium through at least two receptors that are expressed on the cell surface. The first is kinase insert domain-containing receptor (KDR)/fetal liver kinase 1 (Flk-1), and the second is FLT-1 (Warren et al., J Clin Invest 95(4): 1789-97 (1995)). These two receptors have different affinities for VEGF and appear to have different cellular responses. See, Athanassiades et al., Placenta 19(7): 465-73 (1998); Li et al. Cell Res 9(1): 11-25 (1999). FLT-1 null mice die in the embryonic stage, at about day 8.5, whereas KDR null mice survive through birth and retain endothelial and hematopoietic cell development. Activation of KDR leads to mitogenesis and to up-regulation of e-nitric oxide synthase (eNOS) and inducible NOS, enzymes in the nitric oxide pathway that contribute to regulation of vasodilation and that play a role in vascular tumor development.

It has been also been reported that VEGF acts as a survival factor for newly formed blood vessels. In the developing retina, for example, vascular regression in response to hyperoxia has been correlated with inhibition of VEGF release by glial cells. See, Alon et al, Nat Med 1 (10): 1024-8(1995). Furthermore, administration of anti-VEGF monoclonal antibodies results in regression of already established tumor-associated vasculature in xenograft models. See, Yuan, et al., Proc Natl Acad Sci USA 93(25): 14765-70(1996). Therefore, antibodies to FCTRX polypeptides may also be used to induce or promote regression of newly formed blood vessels.

Tumor cells additionally respond to hypoxia by secreting VEGF. This response promotes neovascularization and consequently permits tumor growth. Furthermore, it has been found that several tumor cells, including hematopoietic cells (Bellamy et al., Cancer Res 59(3): 728-33 (1999)), breast cancer cells (Speirs et al., Br J Cancer 80(5-6): 898-903(1999)), and Kaposi's sarcoma (Masood et al., Proc Natl Acad Sci USA 94(3): 979-84 (1997)), express the KDR receptor. Such results suggest that in these tumors VEGF is acting not only in a paracrine fashion to stimulate angiogenesis, but also via an autocrine mechanism as well to stimulate proliferation and/or survival of endothelial cells, and/or promoting survival of tumor cells. Accordingly, modulation of angiogenesis by FCTRX antibodies, or other antagonists of FCTRX nucleic acid or polypeptide function, can be used in anoxia-associated conditions to inhibit endothelial cell proliferation, and/or tumor cells such as hematopoietic cells, breast cancer cells, and Kaposi's sarcoma cells.

The similarity between FCTRX polypeptides and VEGF polypeptides suggests that FCTRX nucleic acids and their encoded polypeptides can be used to modulate cell survival. It has been reported that VEGF signaling is important for cell survival. Binding of VEGF to its receptor, VEGF receptor-2 (VEGFR-2/Flk1/KDR), is reported to induce the formation of a complex of VE-cadherin, β-catenin, phosphoinositide-3-OH kinase (PI3-K), and KDR. PI3-K in this complex activates the serine/threonine protein kinase Akt (protein kinase B) by phosphorylation. See, Carmeliet et al., 1999 Cell 98(2): 147-57. Activated Akt is then thought to be necessary and sufficient to mediate the VEGF-dependent survival signal. See, Gerber et al. 1998 J. Biol. Chem. 273(46): 30336-43. These findings indicate that there is a relationship between VEGF signaling and cell survival.

FCTRX nucleic acids according to the invention can be used to identify various cell types, including cancerous cells. For example, Example 7 illustrates that clone 30664188.0.99 (SEQ ID NO:1) is strongly expressed specifically in CNS cancer, lung cancer and ovarian cancer. It is also shown in the Examples that SEQ ID NO:1 produces a gene product which either persists intact in conditioned medium arising from transfecting HEK 293 cells, or is proteolytically cleaved. Evidence presented in Example 13 suggests that the form of the 30664188.0.99 protein (SEQ ID NO:2) that is active in the experiments reported in the Examples is a proteolysis product of the 30664188.0.99 protein. The activities ascribed to either one or both of these substances include the ability to stimulate net DNA synthesis as monitored by incorporation of BrdU into DNA, proliferation of cell number, the ability to transform cells in culture, and the ability to induce tumor formation in vivo. These various activities occur in a variety of cell types.

A FCTRX nucleic acid or gene product, e.g., a nucleic acid encoding SEQ ID NO:2 or SEQ ID NO:4, is useful as a therapeutic agent in promoting wound healing, neovascularization and tissue growth, and similar tissue regeneration needs. More specifically, a FCTRX nucleic acid or polypeptide may be useful in treatment of anemia and leukopenia, intestinal tract sensitivity and baldness. Treatment of such conditions may be indicated in, e.g., patients having undergone radiation or chemotherapy. It is intended in such cases that administration of a FCTX nucleic acid or polypeptide, e.g., a polypeptide including the amino acid sequence of SEQ ID NO:2 or SEQ ID NO:4, or a nucleic acid sequence encoding these polypeptides (e.g., SEQ ID NO:1 or SEQ ID NO:3) will be controlled in dose such that any hyperproliferative side effects are minimized.

Alternatively, in cases of tumors, such as CNS cancer and ovarian cancer, in which FCTRX nucleic acids is expressed at high levels, (e.g., a tumor in SEQ ID NO:1 is expressed in high levels), it is desired to inhibit or eliminate the effects of production of a FCTRX nucleic acid or gene product (e.g., SEQ ID NO:2 or SEQ ID NO:4, or a nucleic acid encoding one of these polypeptides). For example, this may be accomplished by administration of an antibody directed against a polypeptide having the amino acid sequence of SEQ ID NO:2 or SEQ ID NO:4, or fragment thereof. In particular, the antibody can be directed against the active fragment p35 (see the Examples) identified herein. An alternative example involves identifying the putative protease implicated in the formation of p35 from p85 (see the Examples). Administration of a substance that specifically inhibits the activity of this protease, but not the activity of other proteases, will be effective to prevent formation of the active p35 form of a FCTRX polypeptide, e.g., a clone 30664188.0.99 polypeptide.

Based on the roles of molecules related to FCTRX polypeptides and nucleic acids, (e.g., BMP-1 and VEGF-like polypeptides such as fallotein) in malignant disease progression and the gene expression profile described herein, it is foreseen that, for a subset of human gliomas and ovarian epithelial carcinomas, targeting of a FCTRX polypeptide using an antibody has an inhibitory effect on tumor growth, matrix invasion, chemo-resistance, radio-resistance, and metastatic dissemination. In various embodiments, the FCTRX polypeptide is linked to a monoclonal antibody, a humanized antibody or a fully human antibody.

A FCTRX polypeptide can potentially block or limit the extent of tumor neovascularization. In addition to classical modes of administration of potential antibody therapeutics newly developed modalities of administration may be useful. For example, local administration of ¹³¹I-labeled monoclonal antibody for treatment of primary brain tumors after surgical resection has been reported. Additionally, direct stereotactic intracerebral injection of monoclonal antibodies and their fragments is also being studied clinically and pre-clinically. Intracarotid hyperosmolar perfusion is an experimental strategy to target primary brain malignancy with drug conjugated human monoclonal antibodies.

Additionally, the nucleic acids of the invention, and fragments and variants thereof, may be used, by way of nonlimiting example, (a) to direct the biosynthesis of the corresponding encoded proteins, polypeptides, fragments and variants as recombinant or heterologous gene products, (b) as probes for detection and quantification of the nucleic acids disclosed herein, (c) as sequence templates for preparing antisense molecules, and the like. Such uses are described more fully in the following disclosure.

Furthermore, the proteins and polypeptides of the invention, and fragments and variants thereof, may be used, in ways that include (a) serving as an immunogen to stimulate the production of an anti-FCTRX antibody, (b) a capture antigen in an immunogenic assay for such an antibody, and (c) as a target for screening for substances that bind to a FCTRX polypeptide of the invention. These utilities and other utilities for FCTRX nucleic acids, polypeptides, antibodies, agonists, antagonists, and other related compounds uses are disclosed more fully below. In view of its strong effects in modulating cell growth, an increase of FCTRX polypeptide expression or activity can be used to promote cell survival. Conversely, a decrease in FCTRX polypeptide expression can be used to induce cell death.

FCTRX Nucleic Acids

The novel nucleic acids of the invention include those that encode a FCTRX polypeptide or biologically active portions thereof. The nucleic acids include nucleic acids encoding FCTRX polypeptides that include the amino acid sequence of one or more of SEQ ID NOS:2 and 4. In some embodiments, a nucleic acid encoding a polypeptide having the amino acid sequence of one or more of SEQ ID NOS:2 and 4 includes the nucleic acid sequence of any of SEQ ID NOS:1 and 3, or a fragment thereof.

Additionally, a FCTRX nucleic acid of the invention includes mutant or variant nucleic acids of any of SEQ ID NOS:1 and 3, or a fragment thereof, any of whose bases may be changed from the disclosed sequence while still encoding a protein that maintains its FCTRX-like activities and physiological functions. The invention further includes the complement of the nucleic acid sequence of any of SEQ ID NOS:1 and 3, including fragments, derivatives, analogs and homolog thereof. The invention additionally includes nucleic acids or nucleic acid fragments, or complements thereto, whose structures include chemical modifications.

A FCTRX nucleic acid of the invention can encode a mature form of a FCTRX polypeptide. As used herein, a “mature” form of a polypeptide or protein is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Additionally, a “mature” protein or fragment may arise from a cleavage event other than removal of an initiating methionine or removal of a signal peptide. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

Also included are nucleic acid fragments sufficient for use as hybridization probes to identify nucleic acids encoding FCTRX polypeptides (e.g., a FCTRX mRNA encoding SEQ ID NO:2 or SEQ ID NO:4) and fragments for use as polymerase chain reaction (PCR) primers for the amplification or mutation of FCTRX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source (although they may be prepared by chemical synthesis as well), are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated FCTRX nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NOS:1 and 3, or a complement of any of this nucleotide sequence, can be isolated using standard molecular biology techniques and the sequence information provided herein. Using all or a portion of the nucleic acid sequence of any of SEQ ID NOS:1 and 3 as a hybridization probe, FCTRX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to FCTRX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of any of SEQ ID NOS:1 and 3, or a complement thereof. Oligonucleotides may be chemically synthesized and may be used as probes.

In another embodiment, an isolated nucleic acid molecule of the invention comprises a nucleic acid molecule that is a complement of the nucleotide sequence shown in any of SEQ ID NOS:1 and 3. In another embodiment, an isolated nucleic acid molecule of the invention comprises a nucleic acid molecule that is a complement of the nucleotide sequence shown in any of SEQ ID NOS:1 and 3, or a portion of this nucleotide sequence. A nucleic acid molecule that is complementary to the nucleotide sequence shown in is one that is sufficiently complementary to the nucleotide sequence shown in of any of SEQ ID NOS:1 and 3 that it can hydrogen bond with little or no mismatches to the nucleotide sequence shown in of any of SEQ ID NOS:1 and 3, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of any of SEQ ID NOS:1 and 3, e.g., a fragment that can be used as a probe or primer, or a fragment encoding a biologically active portion of a FCTRX polypeptide. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which is incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a FCTRX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a FCTRX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human FCTRX protein. Homologous nucleic acid sequences include those nucleic acid sequences that encode conservative amino acid substitutions (see below) in any of SEQ ID NOS:2 and 4 as well as a polypeptide having FCTRX activity. Biological activities of the FCTRX proteins are described herein.

As used herein, “identical” residues correspond to those residues in a comparison between two sequences where the equivalent nucleotide base or amino acid residue in an alignment of two sequences is the same residue. Residues are alternatively described as “similar” or “positive” when the comparisons between two sequences in an alignment show that residues in an equivalent position in a comparison are either the same amino acid or a conserved amino acid as defined below.

The nucleotide sequence determined from the cloning of the human FCTRX gene allows for the generation of probes and primers designed for use in identifying the cell types disclosed and/or cloning FCTRX protein homologues in other cell types, e.g., from other tissues, as well as FCTRX homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NOS:1 and 3; or an anti-sense strand nucleotide sequence of SEQ ID NOS:1 and 3; or of a naturally occurring mutant of SEQ ID NOS:1 and 3.

Probes based on a human FCTRX nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a FCTRX protein, such as by measuring a level of a FCTRX protein-encoding nucleic acid in a sample of cells from a subject e.g., detecting mRNA levels or determining whether a genomic FCTRX gene has been mutated or deleted.

“A polypeptide having a biologically active portion of a FCTRX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of a FCTRX polypeptide” can be prepared by isolating a portion of SEQ ID NOS:1 or 3 that encodes a polypeptide having a FCTRX polypeptide biological activity such as those disclosed herein, expressing the encoded portion of FCTRX protein (e.g., by recombinant expression in vitro) and assessing the activity of the encoded portion of the FCTRX polypeptide.

FCTRX Variants

The invention further encompasses nucleic acid molecules that differ from the disclosed FCTRX nucleotide sequences due to degeneracy of the genetic code. These nucleic acids thus encode the same FCTRX protein as that encoded by the nucleotide sequence shown in SEQ ID NOS:1 and 3. In another embodiment, an isolated nucleic acid molecule of the invention has a nucleotide sequence encoding a protein having an amino acid sequence shown in any of SEQ ID NOS:2 and 4.

In addition to the human FCTRX nucleotide sequence shown in any of SEQ ID NOS:1 and 3, it will be appreciated by those skilled in the art that DNA sequence polymorphisms that lead to changes in the amino acid sequences of a FCTRX may exist within a population (e.g., the human population). Such genetic polymorphism in the FCTRX gene may exist among individuals within a population due to natural allelic variation. As used herein, the terms “gene” and “recombinant gene” refer to nucleic acid molecules comprising an open reading frame encoding a FCTRX protein, preferably a mammalian FCTRX protein. Such natural allelic variations can typically result in 1-5% variance in the nucleotide sequence of the FCTRX gene. Any and all such nucleotide variations and resulting amino acid polymorphisms in the FCTRX gene that are the result of natural allelic variation and that do not alter the functional activity of the FCTRX polypeptide are intended to be within the scope of the invention.

Moreover, nucleic acid molecules encoding FCTRX proteins from other species, and thus that have a nucleotide sequence that differs from the human sequence of any of SEQ ID NOS:1 and 3, are intended to be within the scope of the invention. Nucleic acid molecules corresponding to natural allelic variants and homologues of the FCTRX cDNAs of the invention can be isolated based on their homology to the human FCTRX nucleic acids disclosed herein using the human cDNAs, or a portion thereof, as a hybridization probe according to standard hybridization techniques under stringent hybridization conditions.

In another embodiment, an isolated nucleic acid molecule of the invention is at least 6 nucleotides in length and hybridizes under stringent conditions to the nucleic acid molecule comprising the nucleotide sequence of any of SEQ ID NOS:1 and 3. In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences that exceed a minimum degree of similarity to each other typically remain hybridized to each other. For example, depending on the degree of stringency imposed, nucleotide sequences at least about 60% similar to each other may hybridize.

As used herein, the phrase “stringent hybridization conditions” refers to conditions under which a probe, primer or oligonucleotide will hybridize to a target sequence; optimally the probe will hybridize to no other sequences, and more generally will not hybridize to sequences below a specified degree of similarity to the probe. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present at excess, at Tₘ, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions such as described above are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% identical to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of any of SEQ ID NOS:1 and 3 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

Homologs (i.e., nucleic acids encoding FCTRX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

In a second embodiment, a nucleic acid sequence that is hybridizable to the nucleic acid molecule comprising the nucleotide sequence of any of SEQ ID NOS:1 and 3, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of any of SEQ ID NOS:1 and 3, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Conservative Mutations

In addition to naturally-occurring allelic variants of a FCTRX nucleotide sequence, e.g., a gene sequence, that may exist in the population, the skilled artisan will further appreciate that changes can be introduced by mutation into the nucleotide sequence of any of SEQ ID NOS:1 and 3, thereby leading to changes in the amino acid sequence of the encoded FCTRX protein, without altering the functional ability of the FCTRX protein. For example, nucleotide substitutions leading to amino acid substitutions at “non-essential” amino acid residues can be made in the sequence of any of SEQ ID NOS:1 and 3. A “non-essential” amino acid residue is a residue at a position in the sequence that can be altered from the wild-type sequence of the FCTRX polypeptide without altering the biological activity, whereas an “essential” amino acid residue is a residue at a position that is required for biological activity. For example, amino acid residues that are conserved among members of a family of FCTRX proteins, of which the FCTRX proteins of the present invention are members, are predicted to be particularly unamenable to alteration.

For example, a FCTRX protein according to the present invention can contain at least one domain that is a typically conserved region in a FCTRX protein family member. As such, these conserved domains are not likely to be amenable to mutation. Other amino acid residues, however, (e.g., those that are poorly conserved among members of the FCTRX protein family) may not be as essential for activity and thus are more likely to be amenable to alteration.

Another aspect of the invention pertains to nucleic acid molecules encoding FCTRX proteins that contain changes in amino acid residues relative to the amino acid sequence of SEQ ID NO:2 or SEQ ID NO:4 that are not essential for activity. In one embodiment, the isolated nucleic acid molecule comprises a nucleotide sequence encoding a protein, wherein the protein comprises an amino acid sequence at least about 75% similar to the amino acid sequence of any of SEQ ID NOS:2 and 4. Preferably, the protein encoded by the nucleic acid is at least about 80% identical to any of SEQ ID NOS:2 and 4, more preferably at least about 90%, 95%, 98%, and most preferably at least about 99% identical to SEQ ID NO:2.

An isolated nucleic acid molecule encoding a protein homologous to the protein of any of SEQ ID NOS:2 and 4 can be created by introducing one or more nucleotide substitutions, additions or deletions into the corresponding nucleotide sequence, such that one or more amino acid substitutions, additions or deletions are introduced into the encoded protein.

Mutations can be introduced into SEQ ID NOS:1 and 3 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. Certain amino acids have side chains with more than one classifiable characteristic. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, tryptophan, cysteine), nonpolar side chains (e.g., alanine, valine, leucine, isoleucine, proline, phenylalanine, methionine, tyrosine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a FCTRX polypeptide is replaced with another amino acid residue from the same side chain family. Alternatively, in another embodiment, mutations can be introduced randomly along all or part of a FCTRX coding sequence, such as by saturation mutagenesis, and the resultant mutants can be screened for FCTRX polypeptide biological activity to identify mutants that retain activity. Following mutagenesis of SEQ ID NOS:1 and 3 the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY.

In one embodiment, a mutant FCTRX polypeptide can be assayed for (1) the ability to form protein:protein interactions with other FCTRX proteins, other cell-surface proteins, or biologically active portions thereof, (2) complex formation between a mutant FCTRX protein and a FCTRX receptor; (3) the ability of a mutant FCTRX protein to bind to an intracellular target protein or biologically active portion thereof; (e.g., avidin proteins); (4) the ability to bind BRA protein; or (5) the ability to specifically bind an antibody to a FCTRX polypeptide.

In other embodiments, a mutant FCTRX protein can be assayed for its ability to induce tumor formation, or to transform cells, such as NIH 3T3 cells, as described in the Examples below.

Antisense FCTRX Nucleic Acids

Another aspect of the invention pertains to isolated antisense nucleic acid molecules that are hybridizable to or complementary to a FCTRX nucleic acid, e.g., the antisense nucleic acid can be complementary to a nucleic acid molecule comprising the nucleotide sequence of SEQ ID NOS:1 and 3, or fragments, analogs or derivatives thereof. An “antisense” nucleic acid includes a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire FCTRX coding strand, or to only a portion thereof. Nucleic acid molecules encoding fragments, homologs, derivatives and analogs of a FCTRX protein of any of SEQ ID NOS:2 and 4 or antisense nucleic acids complementary to a FCTRX nucleic acid sequence of SEQ ID NOS:1 and 3 are additionally provided.

In one embodiment, an antisense nucleic acid molecule is antisense to a “coding region” of the coding strand of a nucleotide sequence encoding a FCTRX polypeptide. The term “coding region” refers to the region of the nucleotide sequence comprising codons which are translated into amino acid residues (e.g., the protein coding region of a FCTRX polypeptide that corresponds to any of SEQ ID NOS:2 and 4). In another embodiment, the antisense nucleic acid molecule is antisense to a “noncoding region” of the coding strand of a nucleotide sequence encoding a FCTRX polypeptide. The term “noncoding region” refers to 5′ and 3′ sequences which flank the coding region that are not translated into amino acids (i.e., also referred to as 5′ and 3′ untranslated regions).

The FCTRX coding strand sequences disclosed herein (e.g., SEQ ID NOs:1 and 3) allow for antisense nucleic acids to be designed according to the rules of Watson and Crick or Hoogsteen base pairing. The antisense nucleic acid molecule can be complementary to the entire coding region of a FCTRX mRNA. Alternatively, the antisense nucleic acid molecule can be an oligonucleotide that is antisense to only a portion of the coding or noncoding region of a FCTRX mRNA. For example, the antisense oligonucleotide can be complementary to the region surrounding the translation start site of the FCTRX mRNA. An antisense oligonucleotide can be, for example, about 5, 10, 15, 20, 25, 30, 35, 40, 45 or 50 nucleotides in length.

An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a FCTRX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are generally preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-O-methylribonucleotide (Inoue et al. (1987) Nucleic Acids Res 15: 6131-6148) or a chimeric RNA-DNA analogue (Inoue et al. (1987) FEBS Lett 215: 327-330).

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

Also within the invention is a FCTRX ribozymes. Ribozymes are catalytic RNA molecules with ribonuclease activity that are capable of cleaving a single-stranded nucleic acid, such as a FCTRX mRNA, to which they have a complementary region. Thus, ribozymes (e.g., hammerhead ribozymes (described in Haselhoff and Gerlach (1988) Nature 334: 585-591)) can be used to catalytically cleave the FCTRX mRNA transcripts to thereby inhibit translation of the FCTRX mRNA. A ribozyme having specificity for a FCTRX-encoding nucleic acid can be designed based upon the nucleotide sequence of a FCTRX nucleic acid disclosed herein (i.e., SEQ ID NOS:1 and 3). For example, a derivative of a Tetrahymena L-19 IVS RNA can be constructed in which the nucleotide sequence of the active site is complementary to the nucleotide sequence to be cleaved in a FCTRX-encoding mRNA. See, e.g., Cech et al., U.S. Pat. No. 4,987,071; and Cech et al. U.S. Pat. No. 5,116,742. Alternatively, a FCTRX mRNA can be used to select a catalytic RNA having a specific ribonuclease activity from a pool of RNA molecules. See, e.g., Bartel et al., (1993) Science 261: 1411-1418.

Alternatively, FCTRX gene expression can be inhibited by targeting nucleotide sequences complementary to the regulatory region of a FCTRX gene (e.g., the FCTRX gene promoter and/or enhancers) to form triple helical structures that prevent transcription of the FCTRX gene in target cells. See generally, Helene. (1991) Anticancer Drug Des. 6: 569-84; Helene. et al. (1992) Ann. N.Y. Acad. Sci. 660: 27-36; and Maher (1992) Bioassays 14: 807-15.

In various embodiments, the FCTRX nucleic acids can be modified at the base moiety, sugar moiety or phosphate backbone to improve, e.g. the stability, hybridization, or solubility of the molecule. For example, the deoxyribosephosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribosephosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) Proc. Nat. Acad. Sci. (USA) 93: 14670-675.

PNAs based on FCTRX nucleic acids can be used in therapeutic and diagnostic applications. For example, PNAs can be used as antisense or antigene agents for sequence-specific modulation of gene expression by, e.g., inducing transcription or translation arrest or inhibiting replication. PNA based on FCTRX nucleic acids can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In a further embodiment, PNAs of FCTRX nucleic acids can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of the nucleic acids can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, a FCTRX nucleic acid or antisense nucleic acid may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84: 648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6: 958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

FCTRX Polypeptides

A FCTRX polypeptide of the invention includes a protein whose sequence is provided in SEQ ID NO:2 or 4. The invention also includes a mature form of a FCTRX polypeptide, as well as a mutant or variant form of a FCTRX polypeptide. In some embodiments, a mutant or variant FCTRX includes a protein in which any residues may be changed from the corresponding residue shown in FIG. 1, while still encoding a protein that maintains its FCTRX-like activities and physiological functions, or a functional fragment thereof. The invention includes the polypeptides encoded by the variant FCTRX nucleic acids described above. In the mutant or variant protein, up to 20% or more of the residues may be so changed.

In general, a FCTRX polypeptide variant that preserves FCTRX function includes any FCTRX polypeptide variant in which residues at a particular position in the sequence have been substituted by other amino acids. A FCTRX variant polypeptide also includes a FCTRX polypeptide in which an additional residue or residues has been inserted between two residues of the parent protein as well as a protein in which one or more residues have been deleted from a reference FCTRX polypeptide sequence (e.g., SEQ ID NO:2 or SEQ ID NO:4, or a mature form of SEQ ID NO:2 or SEQ ID NO:4). Thus, any amino acid substitution, insertion, or deletion with respect to a reference FCTRX polypeptide sequence (e.g., SEQ ID NO:2 or SEQ ID NO:4, or a mature form of SEQ ID NO:2 or SEQ ID NO:4) is encompassed by the invention. In some embodiments, a mutant or variant proteins may include one or more substitutions, insertions, or deletions with respect to a reference FCTRX sequence.

The invention also includes isolated FCTRX proteins, and biologically active portions thereof, or derivatives, fragments, analogs or homologs thereof. Also provided are polypeptide fragments suitable for use as immunogens to raise anti-FCTRX antibodies. In one embodiment, native FCTRX proteins can be isolated from cells or tissue sources by an appropriate purification scheme using standard protein purification techniques. In another embodiment, FCTRX proteins are produced by recombinant DNA techniques. Alternative to recombinant expression, a FCTRX protein or polypeptide can be synthesized chemically using standard peptide synthesis techniques.

An “isolated” or “purified” protein or biologically active portion thereof is substantially free of cellular material or other contaminating proteins from the cell or tissue source from which the FCTRX protein is derived, or substantially free from chemical precursors or other chemicals when chemically synthesized. The language “substantially free of cellular material” includes preparations of a FCTRX protein in which the protein is separated from cellular components of the cells from which it is isolated or recombinantly produced. In one embodiment, the language “substantially free of cellular material” includes preparations of a FCTRX protein having less than about 30% (by dry weight) of non-FCTRX protein (also referred to herein as a “contaminating protein”), more preferably less than about 20% of non-FCTRX protein, still more preferably less than about 10% of non-FCTRX protein, and most preferably less than about 5% non-FCTRX protein. When the FCTRX protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of a FCTRX protein in which the protein is separated from chemical precursors or other chemicals that are involved in the synthesis of the protein. In one embodiment, the language “substantially free of chemical precursors or other chemicals” includes preparations of a FCTRX protein having less than about 30% (by dry weight) of chemical precursors or non FCTRX polypeptides, more preferably less than about 20% chemical precursors or non-FCTRX polypeptides, still more preferably less than about 10% chemical precursors or non-FCTRX polypeptides, and most preferably less than about 5% chemical precursors or non-FCTRX polypeptides.

Biologically active portions of a FCTRX protein include peptides comprising amino acid sequences sufficiently homologous to or derived from the amino acid sequence of the FCTRX protein, e.g., the amino acid sequence shown in SEQ ID NO:2 that include fewer amino acids than the full length FCTRX proteins, and exhibit at least one activity of a FCTRX protein. Typically, biologically active portions comprise a domain or motif with at least one activity of the FCTRX protein. A biologically active portion of a FCTRX protein can be a polypeptide which is, for example, 10, 25, 50, 100 or more amino acids in length.

A biologically active portion of a FCTRX of the present invention may contain at least one of the above-identified domains conserved among the FCTRX family of proteins. Moreover, other biologically active portions, in which other regions of the protein are deleted, can be prepared by recombinant techniques and evaluated for one or more of the functional activities of a native FCTRX protein.

In some embodiments, the FCTRX protein is substantially homologous to any of SEQ ID NOS:2 and 4 and retains the functional activity of the protein of any of SEQ ID NOS:2 and 4, yet differs in amino acid sequence due to natural allelic variation or mutagenesis, as described in detail below. Accordingly, in another embodiment, the FCTRX protein is a protein that comprises an amino acid sequence at least about 45% homologous, and more preferably about 55, 65, 70, 75, 80, 85, 90, 95, 98 or even 99% homologous to the amino acid sequence of any of SEQ ID NOS:2 and 4 and retains the functional activity of the FCTRX proteins of the corresponding polypeptide having the sequence of SEQ ID NOS:2 and 4.

Determining Homology Between Two or More Sequences

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1 and 3. Equivalent software procedures for determining the extent of sequence identity are widely known in the art may be used in the present context.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T or U, C, G, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

Chimeric and Fusion FCTRX Proteins

The invention also provides FCTRX chimeric or fusion proteins. As used herein, a FCTRX “chimeric protein” or “fusion protein” includes a FCTRX polypeptide operatively linked to a non-FCTRX polypeptide. A “FCTRX polypeptide” refers to a polypeptide having an amino acid sequence corresponding to a FCTRX polypeptide, or a fragment, variant or derivative thereof, whereas a “non-FCTRX polypeptide” refers to a polypeptide having an amino acid sequence corresponding to a protein that is not substantially homologous to the FCTRX protein, e.g., a protein that is different from the FCTRX protein and that is derived from the same or a different organism. Thus, within a FCTRX fusion protein, the FCTRX polypeptide can correspond to all or a portion of a FCTRX protein. In one embodiment, a FCTRX fusion protein comprises at least one biologically active portion of a FCTRX protein. In another embodiment, a FCTRX fusion protein comprises at least two biologically active portions of a FCTRX protein. Within the fusion protein, the term “operatively linked” is intended to indicate that the FCTRX polypeptide and the non-FCTRX polypeptide are fused in-frame to each other. The non-FCTRX polypeptide can be fused to the N-terminus or C-terminus of the FCTRX polypeptide.

For example, in one embodiment a FCTRX fusion protein comprises a FCTRX polypeptide operably linked to the extracellular domain of a second protein. Such fusion proteins can be further utilized in screening assays for compounds that modulate FCTRX activity (such assays are described in detail below).

In another embodiment, the fusion protein is a GST-FCTRX fusion protein in which the FCTRX sequences are fused to the C-terminus of the GST (i.e., glutathione S-transferase) sequences. Such fusion proteins can facilitate the purification of recombinant FCTRX.

In yet another embodiment, the fusion protein is a FCTRX protein containing a heterologous signal sequence at its N-terminus. For example, the native FCTRX signal sequence can be removed and replaced with a signal sequence from another protein. In certain host cells (e.g., mammalian host cells), expression and/or secretion of the FCTRX can be increased through use of a heterologous signal sequence.

In a further embodiment, the fusion protein is a FCTRX-immunoglobulin fusion protein in which the FCTRX sequences comprising one or more domains are fused to sequences derived from a member of the immunoglobulin protein family. The FCTRX-immunoglobulin fusion proteins of the invention can be incorporated into pharmaceutical compositions and administered to a subject to inhibit an interaction between a FCTRX ligand and a FCTRX protein on the surface of a cell, to thereby suppress FCTRX-mediated signal transduction in vivo. In one example, a contemplated FCTRX ligand of the invention is a FCTRX receptor. The FCTRX-immunoglobulin fusion proteins can be used to modulate the bioavailability of a FCTRX cognate ligand. Inhibition of the FCTRX ligand/FCTRX interaction may be useful therapeutically for both the treatment of proliferative and differentiative disorders, as well as modulating (e.g., promoting or inhibiting) cell survival. Moreover, the FCTRX-immunoglobulin fusion proteins of the invention can be used as immunogens to produce anti-FCTRX antibodies in a subject, to purify FCTRX ligands, and in screening assays to identify molecules that inhibit the interaction of a FCTRX with a FCTRX ligand. A FCTRX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A FCTRX-encoding nucleic acid can be cloned into such an expression vector such that the fusion moiety is linked in-frame to the FCTRX protein.

FCTRX Agonists and Antagonists

The present invention also pertains to variants of a FCTRX protein that function as either FCTRX agonists (mimetics) or as FCTRX antagonists. Variants of a FCTRX protein can be generated by mutagenesis, e.g., discrete point mutation or truncation of the FCTRX protein. An agonist of the FCTRX protein can retain substantially the same, or a subset of, the biological activities of the naturally occurring form of the FCTRX protein. An antagonist of the FCTRX protein can inhibit one or more of the activities of the naturally occurring form of the FCTRX protein by, for example, competitively binding to a downstream or upstream member of a cellular signaling cascade which includes the FCTRX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the FCTRX protein.

Variants of the FCTRX protein that function as either FCTRX agonists (mimetics) or as FCTRX antagonists can be identified by screening combinatorial libraries of mutants, e.g., truncation mutants, of the FCTRX protein for FCTRX protein agonist or antagonist activity. In one embodiment, a variegated library of FCTRX variants is generated by combinatorial mutagenesis at the nucleic acid level and is encoded by a variegated gene library. A variegated library of FCTRX variants can be produced by, for example, enzymatically ligating a mixture of synthetic oligonucleotides into gene sequences such that a degenerate set of potential FCTRX sequences is expressible as individual polypeptides, or alternatively, as a set of larger fusion proteins (e.g., for phage display) containing the set of FCTRX sequences therein. There are a variety of methods which can be used to produce libraries of potential FCTRX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential FCTRX variant sequences. Methods for synthesizing degenerate oligonucleotides are known in the art (see, e.g., Narang (1983) Tetrahedron 39: 3; Itakura et al. (1984) Annu Rev Biochem 53: 323; Itakura et al. (1984) Science 198: 1056; Ike et al. (1983) Nucl Acid Res 11: 477.

Polypeptide Libraries

In addition, libraries of fragments of the FCTRX protein coding sequence can be used to generate a variegated population of growth promoter fragments for screening and subsequent selection of variants of a FCTRX protein. In one embodiment, a library of coding sequence fragments can be generated by treating a double stranded PCR fragment of a FCTRX coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the FCTRX protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of FCTRX proteins. The most widely used techniques, which are amenable to high throughput analysis, for screening large gene, libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify FCTRX variants (Arkin and Yourvan (1992) PNAS 89: 7811-7815; Delgrave et al. (1993) Protein Engineering 6: 327-331).

Anti-FCTRX Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′}and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NOs:2 and 4, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of the FCTRX that is located on the surface of the protein, e.g., a hydrophilic region. A hydrophobicity analysis of the human FCTRX protein sequence will indicate which regions of a FCTRX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described in the art. See, e.g., Kohler and Milstein, 1975 Nature, 256: 495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. See, e.g. Kozbor 1984 J. Immunol., 133: 3001; Brodeur et al. MONOCLONAL ANTIBODY PRODUCTION TECHNIQUES AND APPLICATIONS, Marcel Dekker, Inc., New York, (1987) pp. 51-63.

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis. See, e.g. Munson and Pollard 1980 Anal. Biochem. 107: 220. It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2: 593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227: 381 (1991); Marks et al., J. Mol. Biol., 222: 581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See publication WO 94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

Techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305: 537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J. 10: 3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121: 210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90: 6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152: 5368 (1994).

Antibodies with more than two valencies are contemplated. For example, trispecific antibodies can be prepared. Tutt et al., J. Immunol. 147: 60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: THE SCIENCE AND PRACTICE OF PHARMACY 19th ed. (Alfonso R. Gennaro, et al., editors) Mack Pub. Co., Easton, Pa. 1995; DRUG ABSORPTION ENHANCEMENT: CONCEPTS, POSSIBILITIES, LIMITATIONS, AND TRENDS, Harwood Academic Publishers, Langhorne, Pa., 1994; and PEPTIDE AND PROTEIN DRUG DELIVERY (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

FCTRX Recombinant Vectors and Host Cells

Another aspect of the invention pertains to vectors, preferably expression vectors, containing a nucleic acid encoding a FCTRX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., FCTRX proteins, mutant forms of the FCTRX, fusion proteins, etc.).

The recombinant expression vectors of the invention can be designed for expression of a FCTRX nucleic acid in prokaryotic or eukaryotic cells. For example, the FCTRX can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example, using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often a proteolytic cleavage site is introduced in fusion expression vectors at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69: 301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20: 2111-2118). Such alteration of nucleic acid sequences of the invention can be carried out by standard DNA synthesis techniques.

In another embodiment, the FCTRX expression vector is a yeast expression vector. Examples of vectors for expression in yeast S. cerivisae include pYepSec1 (Baldari, et al., (1987) EMBO J6: 229-234), pMFa (Kurjan and Herskowitz, (1982) Cell 30: 933-943), pJRY88 (Schultz et al., (1987) Gene 54: 113-123), pYES2 (Invitrogen Corporation, San Diego, Calif.), and picZ (InVitrogen Corp, San Diego, Calif.).

Alternatively, the FCTRX nucleic acid can be expressed in insect cells using baculovirus expression vectors. Baculovirus vectors available for expression of proteins in cultured insect cells (e.g., SF9 cells) include the pAc series (Smith et al. (1983) Mol Cell Biol 3: 2156-2165) and the pVL series (Lucklow and Summers (1989) Virology 170: 31-39).

In yet another embodiment, a nucleic acid of the invention is expressed in mammalian cells using a mammalian expression vector. Examples of mammalian expression vectors include pCDM8 (Seed (1987) Nature 329: 840) and pMT2PC (Kaufman et al. (1987) EMBO J. 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1: 268-277), lymphoid-specific promoters (Calame and Eaton (1988) Adv Immunol 43: 235-275), in particular promoters of T cell receptors (Winoto and Baltimore (1989) EMBO J. 8: 729-733) and immunoglobulins (Banerji et al. (1983) Cell 33: 729-740; Queen and Baltimore (1983) Cell 33: 741-748), neuron-specific promoters (e.g., the neurofilament promoter; Byrne and Ruddle (1989) PNAS 86: 5473-5477), pancreas-specific promoters (Edlund et al. (1985) Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249: 374-379) and the α-fetoprotein promoter (Campes and Tilghman (1989) Genes Dev 3: 537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to a FCTRX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, the FCTRX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the growth promoter or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) the FCTRX protein. Accordingly, the invention further provides methods for producing the FCTRX protein using the host cells of the invention. In one embodiment, the method comprises culturing the host cell of invention (into which a recombinant expression vector encoding the FCTRX polypeptide has been introduced) in a suitable medium such that the FCTRX protein is produced. In another embodiment, the method further comprises isolating the FCTRX from the medium or the host cell.

Transgenic Animals

The host cells of the invention can also be used to produce nonhuman transgenic animals. For example, in one embodiment, a host cell of the invention is a fertilized oocyte or an embryonic stem cell into which FCTRX-coding sequences have been introduced. Such host cells can then be used to create non-human transgenic animals in which exogenous FCTRX sequences have been introduced into their genome or homologous recombinant animals in which endogenous FCTRX sequences have been altered. Such animals are useful for studying the function and/or activity of the FCTRX sequences and for identifying and/or evaluating modulators of FCTRX activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous FCTRX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing FCTRX-encoding nucleic acid into the male pronuclei of a fertilized oocyte, e.g., by microinjection, retroviral infection, and allowing the oocyte to develop in a pseudopregnant female foster animal. The human FCTRX DNA sequence of SEQ ID NOS:1 and 3 can be introduced as a transgene into the genome of a non-human animal. Alternatively, a nonhuman homologue of the human FCTRX gene, such as a mouse FCTRX gene, can be isolated based on hybridization to the human FCTRX cDNA (described further above) and used as a transgene. Intronic sequences and polyadenylation signals can also be included in the transgene to increase the efficiency of expression of the transgene. A tissue-specific regulatory sequence(s) can be operably linked to the FCTRX transgene to direct expression of FCTRX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the FCTRX transgene in its genome and/or expression of FCTRX mRNA in tissues or cells of the animals. A transgenic founder animal can then be used to breed additional animals carrying the transgene. Moreover, transgenic animals carrying a transgene encoding a FCTRX can further be bred to other transgenic animals carrying other transgenes.

To create a homologous recombinant animal, a vector is prepared which contains at least a portion of a FCTRX gene into which a deletion, addition or substitution has been introduced to thereby alter, e.g., functionally disrupt, the FCTRX gene. The FCTRX gene can be a human gene (e.g., SEQ ID NOS:1 and 3), but more preferably, is a non-human homologue of a human FCTRX gene. For example, a mouse homologue of human FCTRX gene of SEQ ID NOS:1 and 3 can be used to construct a homologous recombination vector suitable for altering an endogenous FCTRX gene in the mouse genome. In one embodiment, the vector is designed such that, upon homologous recombination, the endogenous FCTRX gene is functionally disrupted (i.e., no longer encodes a functional protein; also referred to as a “knock out” vector).

Alternatively, the vector can be designed such that, upon homologous recombination, the endogenous FCTRX gene is mutated or otherwise altered but still encodes functional protein (e.g., the upstream regulatory region can be altered to thereby alter the expression of the endogenous FCTRX protein). In the homologous recombination vector, the altered portion of the FCTRX gene is flanked at its 5′ and 3′ ends by additional nucleic acid of the FCTRX gene to allow for homologous recombination to occur between the exogenous FCTRX protein gene carried by the vector and an endogenous FCTRX protein gene in an embryonic stem cell. The additional flanking FCTRX protein nucleic acid is of sufficient length for successful homologous recombination with the endogenous gene. Typically, several kilobases of flanking DNA (both at the 5′ and 3′ ends) are included in the vector. See e.g., Thomas et al. (1987) Cell 51: 503 for a description of homologous recombination vectors. The vector is introduced into an embryonic stem cell line (e.g., by electroporation) and cells in which the introduced FCTRX protein gene has homologously recombined with the endogenous FCTRX protein gene are selected (see e.g., Li et al. (1992) Cell 69: 915).

The selected cells are then injected into a blastocyst of an animal (e.g., a mouse) to form aggregation chimeras. See e.g., Bradley 1987, In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Curr Opin Biotechnol 2: 823-829; PCT International Publication Nos.: WO 90/1184; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso et al. (1992) PNAS 89: 6232-6236. Another example of a recombinase system is the FLP recombinase system of Saccharomyces cerevisiae (O'Gorman et al. (1991) Science 251: 181-185. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385: 810-813. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Pharmaceutical Compositions

The FCTRX nucleic acid molecules, FCTRX proteins, and anti-FCTRX antibodies of the invention, and derivatives, fragments, analogs and homologs thereof are designated “active compounds” or “Therapeutics” herein. Additionally, low molecular weight compounds which have the property that they either bind to the FCTRX nucleic acid molecules, the FCTRX proteins, and the anti-FCTRX antibodies of the invention, and derivatives, fragments, analogs and homologs thereof, or induce pharmacological agonist or antagonist responses commonly ascribed to a FCTRX nucleic acid molecule, a FCTRX protein, and derivatives, fragments, analogs and homologs thereof, are also termed “active compounds” or “Therapeutics” herein. These Therapeutics can be incorporated into pharmaceutical compositions suitable for administration to a subject. Such compositions typically comprise the nucleic acid molecule, protein, or antibody and a pharmaceutically acceptable carrier.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a FCTRX protein or anti-FCTRX protein antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release pharmaceutical active agents over shorter time periods.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

The nucleic acid molecules of the invention can be inserted into vectors and used as gene therapy vectors. Gene therapy vectors can be delivered to a subject by any of a number of routes, e.g., as described in U.S. Pat. Nos. 5,703,055. Delivery can thus also include, e.g., intravenous injection, local administration (see U.S. Pat. No. 5,328,470) or stereotactic injection (see e.g., Chen et al. (1994) PNAS 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a kit, e.g., in a container, pack, or dispenser together with instructions for administration.

Also within the invention is the use of a therapeutic in the manufacture of a medicament for treating a syndrome associated with a human disease, the disease selected from a FCTRX-associated disorder, wherein said therapeutic is selected from the group consisting of a FCTRX polypeptide, a FCTRX nucleic acid, and an anti-FCTRX antibody.

Additional Uses and Methods of the Invention

The nucleic acid molecules, proteins, protein homologues, and antibodies described herein can be used in one or more of the following methods: (a) screening assays; (b) detection assays (e.g., chromosomal mapping, cell and tissue typing, forensic biology), (c) predictive medicine (e.g., diagnostic assays, prognostic assays, monitoring clinical trials, and pharmacogenomics); and (d) methods of treatment (e.g., therapeutic and prophylactic).

The isolated nucleic acid molecules of the invention can be used to express a FCTRX protein (e.g., via a recombinant expression vector in a host cell in gene therapy applications), to detect a FCTRX mRNA (e.g., in a biological sample) or a genetic lesion in a FCTRX gene, and to modulate FCTRX activity, as described further below. In addition, the FCTRX proteins can be used to screen drugs or compounds that modulate the FCTRX activity or expression as well as to treat disorders characterized by insufficient or excessive production of the FCTRX protein, for example proliferative or differentiative disorders, or production of the FCTRX protein forms that have decreased or aberrant activity compared to the FCTRX wild type protein. In addition, the anti-FCTRX antibodies of the invention can be used to detect and isolate FCTRX proteins and modulate FCTRX activity.

This invention further pertains to novel agents identified by the above described screening assays and uses thereof for treatments as described herein.

Screening Assays

The invention provides methods (also referred to herein as “screening assays”) for identifying modulators, i.e., candidate or test compounds or agents (e.g., proteins, polypeptides, nucleic acids or polynucleotides, peptides, peptidomimetics, small molecules including agonists or antagonists, or other drugs) that bind to FCTRX proteins or have a stimulatory or inhibitory effect on, for example, FCTRX expression or FCTRX activity. The candidate or test compounds or agents that may bind to a FCTRX polypeptide may have a molecular weight around 50 Da, 100 Da, 150 Da, 300 Da, 330 Da, 350 Da, 400 Da, 500 Da, 750 Da, 1000 Da, 1250 Da, 1500 Da, 1750 Da, 2000 Da, 5000 Da, 10,000 Da, 25,000 Da, 50,000 Da, 75,000 Da, 100,000 Da or more than 100,000 Da. In certain embodiments, the candidate substance that binds to a FCTRX polypeptide has a molecular weight not more than about 1500 Da.

Details of functional assays are provided herein further below. Any of the assays described, as well as additional assays known to practitioners in the fields of pharmacology, hematology, internal medicine, oncology and the like, may be employed in order to screen candidate substance for their properties as therapeutic agents. As noted, the therapeutic agents of the invention encompass proteins, polypeptides, nucleic acids or polynucleotides, peptides, peptidomimetics, small molecules including agonists or antagonists, or other drugs described herein.

In one embodiment, the invention provides assays for screening candidate or test compounds which bind to or modulate the activity of a FCTRX protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12: 145).

Examples of methods for the synthesis of molecular libraries can be found in the art, for example in: DeWitt et al. (1993) Proc Natl Acad Sci U.S.A. 90: 6909; Erb et al. (1994) Proc Natl Acad Sci U.S.A. 91: 11422; Zuckermann et al. (1994) J Med Chem 37: 2678; Cho et al. (1993) Science 261: 1303; Carrell et al. (1994) Angew Chem Int Ed Engl 33: 2059; Carell et al. (1994) Angew Chem Int Ed Engl 33: 2061; and Gallop et al. (1994) J Med Chem 37: 1233.

Libraries of compounds may be presented in solution (e.g., Houghten (1992) Biotechniques 13: 412-421), or on beads (Lam (1991) Nature 354: 82-84), on chips (Fodor (1993) Nature 364: 555-556), bacteria (Ladner U.S. Pat. No. 5,223,409), spores (Ladner U.S. Pat. No. '409), plasmids (Cull et al. (1992) Proc Natl Acad Sci USA 89: 1865-1869) or on phage (Scott and Smith (1990) Science 249: 386-390; Devlin (1990) Science 249: 404-406; Cwirla et al. (1990) Proc Natl Acad Sci U.S.A. 87: 6378-6382; Felici (1991) J Mol Biol 222: 301-310; Ladner above.).

In one embodiment, an assay is a cell-based assay in which a cell which expresses a membrane-bound form of a FCTRX protein, or a biologically active portion thereof, on the cell surface is contacted with a test compound and the ability of the test compound to bind to a FCTRX protein determined. The cell, for example, can of mammalian origin or a yeast cell. Determining the ability of the test compound to bind to the FCTRX protein can be accomplished, for example, by coupling the test compound with a radioisotope or enzymatic label such that binding of the test compound to the FCTRX protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of a FCTRX protein, or a biologically active portion thereof, on the cell surface with a known compound which binds a FCTRX to form an assay mixture, contacting the assay mixture with a test compound, and determining the ability of the test compound to interact with a FCTRX protein, wherein determining the ability of the test compound to interact with a FCTRX protein comprises determining the ability of the test compound to preferentially bind to a FCTRX or a biologically active portion thereof as compared to the known compound.

In another embodiment, an assay is a cell-based assay comprising contacting a cell expressing a membrane-bound form of a FCTRX protein, or a biologically active portion thereof, on the cell surface with a test compound and determining the ability of the test compound to modulate (e.g., stimulate or inhibit) the activity of the FCTRX protein or biologically active portion thereof. Determining the ability of the test compound to modulate the activity of a FCTRX polypeptide or a biologically active portion thereof can be accomplished, for example, by determining the ability of the FCTRX protein to bind to or interact with a FCTRX target molecule. As used herein, a “target molecule” is a molecule with which a FCTRX protein binds or interacts in nature, for example, a molecule on the surface of a cell which expresses a FCTRX interacting protein, a molecule on the surface of a second cell, a molecule in the extracellular milieu, a molecule associated with the internal surface of a cell membrane or a cytoplasmic molecule. A FCTRX target molecule can be a non-FCTRX molecule or a FCTRX protein or polypeptide of the present invention. In one embodiment, a FCTRX target molecule is a component of a signal transduction pathway that facilitates transduction of an extracellular signal (e.g., a signal generated by binding of a compound to a membrane-bound FCTRX molecule) through the cell membrane and into the cell. The target, for example, can be a second intercellular protein that has catalytic activity or a protein that facilitates the association of downstream signaling molecules with the FCTRX polypeptide.

Determining the ability of the FCTRX protein to bind to or interact with a FCTRX target molecule can be accomplished by one of the methods described above for determining direct binding. In one embodiment, determining the ability of the FCTRX protein to bind to or interact with a FCTRX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a FCTRX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a FCTRX protein or biologically active portion thereof with a test compound and determining the ability of the test compound to bind to the FCTRX protein or biologically active portion thereof. Binding of the test compound to the FCTRX protein can be determined either directly or indirectly as described above. In one embodiment, the assay comprises contacting the FCTRX protein or biologically active portion thereof with a known compound which binds FCTRX to form an assay mixture, contacting the assay mixture with a test compound, and determining the ability of the test compound to interact with a FCTRX protein, wherein determining the ability of the test compound to interact with a FCTRX protein comprises determining the ability of the test compound to preferentially bind to a FCTRX or biologically active portion thereof as compared to the known compound.

In another embodiment, an assay is a cell-free assay comprising contacting a FCTRX protein or biologically active portion thereof with a test compound and determining the ability of the test compound to modulate (e.g., stimulate or inhibit) the activity of the FCTRX protein or biologically active portion thereof. Determining the ability of the test compound to modulate the activity of a FCTRX polypeptide can be accomplished, for example, by determining the ability of the FCTRX protein to bind to a FCTRX target molecule by one of the methods described above for determining direct binding. In an alternative embodiment, determining the ability of the test compound to modulate the activity of a FCTRX polypeptide can be accomplished by determining the ability of the FCTRX protein further modulate a FCTRX target molecule. For example, the catalytic/enzymatic activity of the target molecule on an appropriate substrate can be determined as previously described.

In yet another embodiment, the cell-free assay comprises contacting the FCTRX protein or biologically active portion thereof with a known compound which binds a FCTRX polypeptide to form an assay mixture, contacting the assay mixture with a test compound, and determining the ability of the test compound to interact with a FCTRX protein, wherein determining the ability of the test compound to interact with a FCTRX protein comprises determining the ability of the FCTRX protein to preferentially bind to or modulate the activity of a FCTRX target molecule.

The cell-free assays of the present invention are amenable to use of both a soluble form or a membrane-bound form of a FCTRX polypeptide. In the case of cell-free assays comprising the membrane-bound form of a FCTRX polypeptide, it may be desirable to utilize a solubilizing agent such that the membrane-bound form of a FCTRX polypeptide is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

It may be desirable to immobilize either a FCTRX polypeptide or its target molecule to facilitate separation of complexed from uncomplexed forms of one or both of the proteins, as well as to accommodate automation of the assay. Binding of a test compound to a FCTRX polypeptide, or interaction of a FCTRX polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-FCTRX polypeptide fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or a FCTRX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of a FCTRX binding or activity determined using standard techniques.

Other techniques for immobilizing proteins on matrices can also be used in the screening assays of the invention. For example, either the FCTRX polypeptide or its target molecule can be immobilized utilizing conjugation of biotin and streptavidin. Biotinylated FCTRX protein or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with FCTRX protein or target molecules, but which do not interfere with binding of the FCTRX protein to its target molecule, can be derivatized to the wells of the plate, and unbound target or FCTRX protein trapped in the wells by antibody conjugation. Methods for detecting such complexes, in addition to those described above for the GST-immobilized complexes, include immunodetection of complexes using antibodies reactive with the FCTRX protein or target molecule, as well as enzyme-linked assays that rely on detecting an enzymatic activity associated with the FCTRX protein or target molecule.

In another embodiment, modulators of a FCTRX expression are identified in a method wherein a cell is contacted with a candidate compound and the expression of a FCTRX mRNA or protein in the cell is determined. The level of expression of a FCTRX mRNA or protein in the presence of the candidate compound is compared to the level of expression of a FCTRX mRNA or protein in the absence of the candidate compound. The candidate compound can then be identified as a modulator of a FCTRX expression based on this comparison. For example, when expression of a FCTRX mRNA or protein is greater (statistically significantly greater) in the presence of the candidate compound than in its absence, the candidate compound is identified as a stimulator of a FCTRX mRNA or protein expression. Alternatively, when expression of a FCTRX mRNA or protein is less (statistically significantly less) in the presence of the candidate compound than in its absence, the candidate compound is identified as an inhibitor of a FCTRX mRNA or protein expression. The level of a FCTRX mRNA or protein expression in the cells can be determined by methods described herein for detecting FCTRX mRNA or protein.

In yet another aspect of the invention, the FCTRX proteins can be used as “bait proteins” in a two-hybrid assay or three hybrid assay (see, e.g., U.S. Pat. No. 5,283,317; Zervos et al. (1993) Cell 72: 223-232; Madura et al. (1993) J Biol Chem 268: 12046-12054; Bartel et al. (1993) Biotechniques 14: 920-924; Iwabuchi et al. (1993) Oncogene 8: 1693-1696; and Brent WO94/10300), to identify other proteins that bind to or interact with the FCTRX (“FCTRX-binding proteins” or “FCTRX-bp”) and modulate FCTRX activity. Such FCTRX-binding proteins are also likely to be involved in the propagation of signals by the FCTRX proteins as, for example, upstream or downstream elements of the FCTRX pathway.

The two-hybrid system is based on the modular nature of most transcription factors, which consist of separable DNA-binding and activation domains. Briefly, the assay utilizes two different DNA constructs. In one construct, the gene that codes for a FCTRX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a FCTRX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with the FCTRX.

Screening can also be performed in vivo. For example, in one embodiment, the invention includes a method for screening for a modulator of activity or of latency or predisposition to a FCTRX-associated disorder by administering a test compound or to a test animal at increased risk for a FCTRX-associated disorder. In some embodiments, the test animal recombinantly expresses a FCTRX polypeptide. Activity of the polypeptide in the test animal after administering the compound is measured, and the activity of the protein in the test animal is compared to the activity of the polypeptide in a control animal not administered said polypeptide. A change in the activity of said polypeptide in said test animal relative to the control animal indicates the test compound is a modulator of latency of or predisposition to a FCTRX-associated disorder.

In some embodiments, the test animal is a recombinant test animal that expresses a test protein transgene or expresses the transgene under the control of a promoter at an increased level relative to a wild-type test animal. Preferably, the promoter is not the native gene promoter of the transgene.

This invention further pertains to novel agents identified by the above-described screening assays and uses thereof for treatments as described herein.

Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample.

The FCTRX sequences of the present invention can also be used to identify individuals from minute biological samples. In this technique, an individual's genomic DNA is digested with one or more restriction enzymes, and probed on a Southern blot to yield unique bands for identification. The sequences of the present invention are useful as additional DNA markers for RFLP (“restriction fragment length polymorphisms,” described in U.S. Pat. No. 5,272,057).

Furthermore, the sequences of the present invention can be used to provide an alternative technique that determines the actual base-by-base DNA sequence of selected portions of an individual's genome. Thus, the FCTRX sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The FCTRX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NOS:1 and 3, as described above, can comfortably provide positive individual identification with a panel of perhaps 10 to 1,000 primers that each yield a noncoding amplified sequence of 100 bases. If predicted coding sequences are used, a more appropriate number of primers for positive individual identification would be 500-2,000.

Use of Partial FCTRX Sequences in Forensic Biology

DNA-based identification techniques based on FCTRX nucleic acid sequences or polypeptide sequences can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, that can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NOS:1 and 3 are particularly appropriate for this use as greater numbers of polymorphisms occur in the noncoding regions, making it easier to differentiate individuals using this technique. Examples of polynucleotide reagents include the FCTRX sequences or portions thereof, e.g., fragments derived from the noncoding regions of one or more of SEQ ID NOS:1 and 3, having a length of at least 20 bases, preferably at least 30 bases.

The FCTRX sequences described herein can further be used to provide polynucleotide reagents, e.g., labeled or label-able probes that can be used, for example, in an in situ hybridization technique, to identify a specific tissue, e.g., brain tissue, etc. This can be very useful in cases where a forensic pathologist is presented with a tissue of unknown origin. Panels of such FCTRX probes can be used to identify tissue by species and/or by organ type.

In a similar fashion, these reagents, e.g., FCTRX primers or probes can be used to screen tissue culture for contamination (i.e. screen for the presence of a mixture of different types of cells in a culture).

Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining a FCTRX protein and/or nucleic acid expression as well as FCTRX activity, in the context of a biological sample (e.g., blood, serum, cells, tissue) to thereby determine whether an individual is afflicted with a disease or disorder, or is at risk of developing a disorder, associated with aberrant FCTRX expression or activity. The invention also provides for prognostic (or predictive) assays for determining whether an individual is at risk of developing a disorder associated with a FCTRX protein, nucleic acid expression or activity. For example, mutations in a FCTRX gene can be assayed in a biological sample. Such assays can be used for prognostic or predictive purpose to thereby prophylactically treat an individual prior to the onset of a disorder characterized by or associated with FCTRX protein, nucleic acid expression or activity.

Another aspect of the invention provides methods for determining FCTRX protein, nucleic acid expression or FCTRX activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of a FCTRX in clinical trials.

These and other agents are described in further detail in the following sections.

Diagnostic Assays

Other conditions in which proliferation of cells plays a role include tumors, restenosis, psoriasis, Dupuytren's contracture, diabetic complications, Kaposi's sarcoma and rheumatoid arthritis.

A FCTRX polypeptide may be used to identify an interacting polypeptide a sample or tissue. The method comprises contacting the sample or tissue with the FCTRX, allowing formation of a complex between the FCTRX polypeptide and the interacting polypeptide, and detecting the complex, if present.

The proteins of the invention may be used to stimulate production of antibodies specifically binding the proteins. Such antibodies may be used in immunodiagnostic procedures to detect the occurrence of the protein in a sample. The proteins of the invention may be used to stimulate cell growth and cell proliferation in conditions in which such growth would be favorable. An example would be to counteract toxic side effects of chemotherapeutic agents on, for example, hematopoiesis and platelet formation, linings of the gastrointestinal tract, and hair follicles. They may also be used to stimulate new cell growth in neurological disorders including, for example, Alzheimer's disease. Alternatively, antagonistic treatments may be administered in which an antibody specifically binding the FCTRX-like proteins of the invention would abrogate the specific growth-inducing effects of the proteins. Such antibodies may be useful, for example, in the treatment of proliferative disorders including various tumors and benign hyperplasias.

Polynucleotides or oligonucleotides corresponding to any one portion of the FCTRX nucleic acids of SEQ ID NOS:1 and 3 may be used to detect DNA containing a corresponding ORF gene, or detect the expression of a corresponding FCTRX gene, or FCTRX-like gene. For example, a FCTRX nucleic acid expressed in a particular cell or tissue, as noted in Table 3, can be used to identify the presence of that particular cell type.

An exemplary method for detecting the presence or absence of a FCTRX polypeptide in a biological sample involves obtaining a biological sample from a test subject and contacting the biological sample with a compound or an agent capable of detecting a FCTRX protein or nucleic acid (e.g., mRNA, genomic DNA) that encodes a FCTRX protein such that the presence of a FCTRX polypeptide is detected in the biological sample. An agent for detecting a FCTRX mRNA or genomic DNA is a labeled nucleic acid probe capable of hybridizing to a FCTRX mRNA or genomic DNA. The nucleic acid probe can be, for example, a full-length FCTRX nucleic acid, such as the nucleic acid of SEQ ID NOS:1 and 3, or a portion thereof, such as an oligonucleotide of at least 15, 30, 50, 100, 250 or 500 nucleotides in length and sufficient to specifically hybridize under stringent conditions to a FCTRX mRNA or genomic DNA, as described above. Other suitable probes for use in the diagnostic assays of the invention are described herein.

An agent for detecting a FCTRX protein is an antibody capable of binding to a FCTRX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect a FCTRX mRNA, protein, or genomic DNA in a biological sample in vitro as well as in vivo. For example, in vitro techniques for detection of a FCTRX mRNA include Northern hybridizations and in situ hybridizations. In vitro techniques for detection of a FCTRX protein include enzyme linked immunosorbent assays (ELISAs), Western blots, immunoprecipitations and immunofluorescence. In vitro techniques for detection of a FCTRX genomic DNA include Southern hybridizations. Furthermore, in vivo techniques for detection of a FCTRX protein include introducing into a subject a labeled anti-FCTRX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a FCTRX protein, mRNA, or genomic DNA, such that the presence of a FCTRX protein, mRNA or genomic DNA is detected in the biological sample, and comparing the presence of a FCTRX protein, mRNA or genomic DNA in the control sample with the presence of a FCTRX protein, mRNA or genomic DNA in the test sample.

The invention also encompasses kits for detecting the presence of a FCTRX polypeptide in a biological sample. For example, the kit can comprise: a labeled compound or agent capable of detecting a FCTRX protein or mRNA in a biological sample; means for determining the amount of a FCTRX polypeptide in the sample; and means for comparing the amount of a FCTRX polypeptide in the sample with a standard. The compound or agent can be packaged in a suitable container. The kit can further comprise instructions for using the kit to detect a FCTRX protein or nucleic acid.

Prognostic Assays

The diagnostic methods described herein can furthermore be utilized to identify subjects having or at risk of developing a disease or disorder associated with aberrant FCTRX polypeptide expression or activity. For example, the assays described herein, such as the preceding diagnostic assays or the following assays, can be utilized to identify a subject having or at risk of developing a disorder associated with a FCTRX protein, nucleic acid expression or activity in, e.g., proliferative or differentiative disorders such as hyperplasias, tumors, restenosis, psoriasis, Dupuytren's contracture, diabetic complications, or rheumatoid arthritis, etc.; and glia-associated disorders such as cerebral lesions, diabetic neuropathies, cerebral edema, senile dementia, Alzheimer's disease, etc. Alternatively, the prognostic assays can be utilized to identify a subject having or at risk for developing a disease or disorder. Thus, the present invention provides a method for identifying a disease or disorder associated with aberrant FCTRX expression or activity in which a test sample is obtained from a subject and a FCTRX protein or nucleic acid (e.g., mRNA, genomic DNA) is detected, wherein the presence of a FCTRX protein or nucleic acid is diagnostic for a subject having or at risk of developing a disease or disorder associated with aberrant FCTRX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant FCTRX expression or activity. For example, such methods can be used to determine whether a subject can be effectively treated with an agent for a disorder, such as a proliferative disorder, differentiative disorder, glia-associated disorders, etc. Thus, the present invention provides methods for determining whether a subject can be effectively treated with an agent for a disorder associated with aberrant FCTRX expression or activity in which a test sample is obtained and a FCTRX protein or nucleic acid is detected (e.g., wherein the presence of a FCTRX protein or nucleic acid is diagnostic for a subject that can be administered the agent to treat a disorder associated with aberrant FCTRX expression or activity.)

The methods of the invention can also be used to detect genetic lesions in a FCTRX gene, thereby determining if a subject with the lesioned gene is at risk for, or suffers from, a proliferative disorder, differentiative disorder, glia-associated disorder, etc. In various embodiments, the methods include detecting, in a sample of cells from the subject, the presence or absence of a genetic lesion characterized by at least one of an alteration affecting the integrity of a gene encoding a FCTRX protein, or the mis-expression of the FCTRX gene. For example, such genetic lesions can be detected by ascertaining the existence of at least one of (1) a deletion of one or more nucleotides from a FCTRX gene; (2) an addition of one or more nucleotides to a FCTRX gene; (3) a substitution of one or more nucleotides of a FCTRX gene, (4) a chromosomal rearrangement of a FCTRX gene; (5) an alteration in the level of a messenger RNA transcript of a FCTRX gene, (6) aberrant modification of a FCTRX gene, such as of the methylation pattern of the genomic DNA, (7) the presence of a non-wild type splicing pattern of a messenger RNA transcript of a FCTRX gene, (8) a non-wild type level of a protein, (9) allelic loss of a FCTRX gene, and (10) inappropriate post-translational modification of a FCTRX protein. As described herein, there are a large number of assay techniques known in the art which can be used for detecting lesions in a FCTRX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241: 1077-1080; and Nakazawa et al. (1994) PNAS 91: 360-364), the latter of which can be particularly useful for detecting point mutations in the FCTRX gene (see Abravaya et al. (1995) Nucl Acids Res 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a FCTRX gene under conditions such that hybridization and amplification of the FCTRX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990, Proc Natl Acad Sci USA 87: 1874-1878), transcriptional amplification system (Kwoh, et al., 1989, Proc Natl Acad Sci USA 86: 1173-1177), Q-Beta Replicase (Lizardi et al, 1988, BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a FCTRX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,493,531) can be used to score for the presence of specific mutations by development or loss of a ribozyme cleavage site.

In other embodiments, genetic mutations in a FCTRX nucleic acid of the invention can be identified by hybridizing a sample and control nucleic acids, e.g., DNA or RNA, to high density arrays containing hundreds or thousands of oligonucleotides probes (Cronin et al. (1996) Human Mutation 7: 244-255; Kozal et al. (1996) Nature Medicine 2: 753-759). For example, genetic mutations in a FCTRX of the invention can be identified in two dimensional arrays containing light-generated DNA probes as described in Cronin et al. above. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the FCTRX gene and detect mutations by comparing the sequence of the sample FCTRX gene with the corresponding wild-type (control) sequence. Examples of sequencing reactions include those based on techniques developed by Maxim and Gilbert (1977) PNAS 74: 560 or Sanger (1977) PNAS 74: 5463. It is also contemplated that any of a variety of automated sequencing procedures can be utilized when performing the diagnostic assays (Naeve et al., (1995) Biotechniques 19: 448), including sequencing by mass spectrometry (see, e.g., PCT International Publ. No. WO 94/16101; Cohen et al. (1996) Adv Chromatogr 36: 127-162; and Griffin et al. (1993) Appl Biochem Biotechnol 38: 147-159).

Other methods for detecting mutations in the FCTRX gene include methods in which protection from cleavage agents is used to detect mismatched bases in RNA/RNA or RNA/DNA heteroduplexes (Myers et al. (1985) Science 230: 1242). In general, the art technique of “mismatch cleavage” starts by providing heteroduplexes of formed by hybridizing (labeled) RNA or DNA containing the wild-type FCTRX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc Natl Acad Sci USA 85: 4397; Saleeba et al (1992) Methods Enzymol 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in FCTRX cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15: 1657-1662). According to an exemplary embodiment, a probe based on a FCTRX sequence, e.g., a wild-type FCTRX sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in FCTRX genes. For example, single strand conformation polymorphism (SSCP) may be used to detect differences in electrophoretic mobility between mutant and wild type nucleic acids (Orita et al. (1989) Proc Natl Acad Sci USA: 86: 2766, see also Cotton (1993) Mutat Res 285: 125-144; Hayashi (1992) Genet Anal Tech Appl 9: 73-79). Single-stranded DNA fragments of sample and control a FCTRX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA, rather than DNA, in which the secondary structure is more sensitive to a change in sequence. In one embodiment, the subject method utilizes heteroduplex analysis to separate double stranded heteroduplex molecules on the basis of changes in electrophoretic mobility. See, e.g., Keen et al. (1991) Trends Genet 7: 5.

In yet another embodiment the movement of mutant or wild-type fragments in polyacrylamide gels containing a gradient of denaturant is assayed using denaturing gradient gel electrophoresis (DGGE). See, e.g., Myers et al (1985) Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner (1987) Biophys Chem 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki et al. (1986) Nature 324: 163); Saiki et al. (1989) Proc Natl Acad. Sci USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res 17: 2437-2448) or at the extreme 3′ end of one primer where, under appropriate conditions, mismatch can prevent, or reduce polymerase extension (Prossner (1993) Tibtech 11: 238). In addition it may be desirable to introduce a novel restriction site in the region of the mutation to create cleavage-based detection. See, e.g., Gasparini et al (1992) Mol Cell Probes 6: 1. It is anticipated that in certain embodiments amplification may also be performed using Taq ligase for amplification. See, e.g., Barany (1991) Proc Natl Acad Sci USA 88: 189. In such cases, ligation will occur only if there is a perfect match at the 3′ end of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a FCTRX gene.

Furthermore, any cell type or tissue, preferably peripheral blood leukocytes, in which a FCTRX of the invention is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on FCTRX activity (e.g., FCTRX gene expression), as identified by a screening assay described herein can be administered to individuals to treat (prophylactically or therapeutically) disorders (e.g., neurological, cancer-related or gestational disorders) associated with aberrant FCTRX activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a FCTRX protein, expression of a FCTRX nucleic acid, or mutation content of a FCTRX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996, Clin Exp Pharmacol Physiol, 23: 983-985 and Linder, 1997, Clin Chem, 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of a FCTRX protein, expression of a FCTRX nucleic acid, or mutation content of a FCTRX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a FCTRX modulator, such as a modulator identified by one of the exemplary screening assays described herein.

Monitoring Clinical Efficacy

Monitoring the influence of agents (e.g., drugs, compounds) on the expression or activity of a FCTRX (e.g., the ability to modulate aberrant cell proliferation and/or differentiation) can be applied in basic drug screening and in clinical trials. For example, the effectiveness of an agent determined by a screening assay as described herein to increase FCTRX gene expression, protein levels, or upregulate FCTRX activity, can be monitored in clinical trials of subjects exhibiting decreased FCTRX gene expression, protein levels, or downregulated FCTRX activity. Alternatively, the effectiveness of an agent determined by a screening assay to decrease FCTRX gene expression, protein levels, or downregulate FCTRX activity, can be monitored in clinical trials of subjects exhibiting increased FCTRX gene expression, protein levels, or upregulated FCTRX activity. In such clinical trials, the expression or activity of a FCTRX and, preferably, other genes that have been implicated in, for example, a proliferative or neurological disorder, can be used as a “read out” or marker of the responsiveness of a particular cell. Other FCTRX-associated disorders include, e.g., cancers, cell proliferation disorders, anxiety disorders; CNS disorders; diabetes; obesity; and infectious disease.

For example, genes, including genes encoding a FCTRX of the invention, that are modulated in cells by treatment with an agent (e.g., compound, drug or small molecule) that modulates a FCTRX activity (e.g., identified in a screening assay as described herein) can be identified. Thus, to study the effect of agents on cellular proliferation disorders, for example, in a clinical trial, cells can be isolated and RNA prepared and analyzed for the levels of expression of a FCTRX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene or other genes. In this way, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, nucleic acid, peptidomimetic, small molecule, or other drug candidate identified by the screening assays described herein) comprising the steps of (i) obtaining a pre-administration sample from a subject prior to administration of the agent; (ii) detecting the level of expression of a FCTRX protein, mRNA, or genomic DNA in the preadministration sample; (iii) obtaining one or more post-administration samples from the subject; (iv) detecting the level of expression or activity of the FCTRX protein, mRNA, or genomic DNA in the post-administration samples; (v) comparing the level of expression or activity of the FCTRX protein, mRNA, or genomic DNA in the pre-administration sample with the FCTRX protein, mRNA, or genomic DNA in the post administration sample or samples; and (vi) altering the administration of the agent to the subject accordingly. For example, increased administration of the agent may be desirable to increase the expression or activity of a FCTRX to higher levels than detected, i.e., to increase the effectiveness of the agent. Alternatively, decreased administration of the agent may be desirable to decrease expression or activity of a FCTRX to lower levels than detected, i.e., to decrease the effectiveness of the agent.

Methods of Treatment

The present invention provides for both prophylactic and therapeutic methods of treating a subject at risk of (or susceptible to) a disorder or having a disorder associated with aberrant FCTRX expression or activity.

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to, (i) a FCTRX polypeptide, or analogs, derivatives, fragments or homologs thereof; (ii) antibodies to a FCTRX peptide; (iii) nucleic acids encoding a FCTRX peptide; (iv) administration of antisense nucleic acid and nucleic acids that are “dysfunctional” (i.e., due to a heterologous insertion within the coding sequences of coding sequences to a FCTRX polypeptide) that are utilized to “knockout” endogenous function of a FCTRX polypeptide by homologous recombination (see, e.g., Capecchi, 1989, Science 244: 1288-1292); or (v) modulators (i.e., inhibitors, agonists and antagonists, including additional peptide mimetic of the invention or antibodies specific to a peptide of the invention) that alter the interaction between a FCTRX peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, a polypeptide, a peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or polypeptide levels, structure and/or activity of the expressed polypeptides (or mRNAs encoding a FCTRX polypeptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

In one aspect, the invention provides a method for preventing, in a subject, a disease or condition associated with aberrant FCTRX expression or activity, by administering to the subject an agent that modulates FCTRX expression or at least one FCTRX activity. Subjects at risk for a disease that is caused or contributed to by aberrant FCTRX expression or activity can be identified by, for example, any or a combination of diagnostic or prognostic assays as described herein. Administration of a prophylactic agent can occur prior to the manifestation of symptoms characteristic of the FCTRX aberrancy, such that a disease or disorder is prevented or, alternatively, delayed in its progression. Depending on the type of a FCTRX aberrancy, for example, a FCTRX agonist or FCTRX antagonist agent can be used for treating the subject. The appropriate agent can be determined based on screening assays described herein.

Another aspect of the invention pertains to methods of modulating FCTRX expression or activity for therapeutic purposes. The modulatory method of the invention involves contacting a cell with an agent that modulates one or more of the activities of a FCTRX protein activity associated with the cell. An agent that modulates a FCTRX protein activity can be an agent as described herein, such as a nucleic acid or a protein, a naturally-occurring cognate ligand of a FCTRX protein, a peptide, a FCTRX peptidomimetic, or other small molecule. In one embodiment, the agent stimulates one or more a FCTRX protein activity. Examples of such stimulatory agents include active a FCTRX protein and a nucleic acid molecule encoding a FCTRX polypeptide that has been introduced into the cell. In another embodiment, the agent inhibits one or more a FCTRX protein activity. Examples of such inhibitory agents include antisense a FCTRX nucleic acid molecules and anti-FCTRX antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a FCTRX protein or nucleic acid molecule. In one embodiment, the method involves administering an agent (e.g., an agent identified by a screening assay described herein), or combination of agents that modulates (e.g., upregulates or downregulates) FCTRX expression or activity. In another embodiment, the method involves administering a FCTRX protein or nucleic acid molecule as therapy to compensate for reduced or aberrant FCTRX expression or activity.

Determination of the Biological Effect of a Therapeutic

In various embodiments of the present invention, suitable in vitro or in vivo assays are utilized to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Malignancies

Some FCTRX polypeptides are expressed in cancerous cells and are therefore implicated in the regulation of cell proliferation. Accordingly, Therapeutics of the present invention may be useful in the therapeutic or prophylactic treatment of diseases or disorders that are associated with cell hyperproliferation and/or loss of control of cell proliferation (e.g., cancers, malignancies and tumors). For a review of such hyperproliferation disorders, see e.g., Fishman, et al., 1985. MEDICINE, 2nd ed., J.B. Lippincott Co., Philadelphia, Pa.

Therapeutics of the present invention may be assayed by any method known within the art for efficacy in treating or preventing malignancies and related disorders. Such assays include, but are not limited to, in vitro assays utilizing transformed cells or cells derived from the patient's tumor, as well as in vivo assays using animal models of cancer or malignancies. Potentially effective Therapeutics are those that, for example, inhibit the proliferation of tumor-derived or transformed cells in culture or cause a regression of tumors in animal models, in comparison to the controls.

In the practice of the present invention, once a malignancy or cancer has been shown to be amenable to treatment by modulating (i.e., inhibiting, antagonizing or agonizing) activity, that cancer or malignancy may subsequently be treated or prevented by the administration of a Therapeutic that serves to modulate protein function.

Premalignant Conditions

The Therapeutics of the present invention that are effective in the therapeutic or prophylactic treatment of cancer or malignancies may also be administered for the treatment of pre-malignant conditions and/or to prevent the progression of a pre-malignancy to a neoplastic or malignant state. Such prophylactic or therapeutic use is indicated in conditions known or suspected of preceding progression to neoplasia or cancer, in particular, where non-neoplastic cell growth consisting of hyperplasia, metaplasia or, most particularly, dysplasia has occurred. For a review of such abnormal cell growth see e.g., Robbins & Angell, 1976. BASIC PATHOLOGY, 2nd ed., W. B. Saunders Co., Philadelphia, Pa.

Hyperplasia is a form of controlled cell proliferation involving an increase in cell number in a tissue or organ, without significant alteration in its structure or function. For example, it has been demonstrated that endometrial hyperplasia often precedes endometrial cancer. Metaplasia is a form of controlled cell growth in which one type of mature or fully differentiated cell substitutes for another type of mature cell. Metaplasia may occur in epithelial or connective tissue cells. Dysplasia is generally considered a precursor of cancer, and is found mainly in the epithelia. Dysplasia is the most disorderly form of non-neoplastic cell growth, and involves a loss in individual cell uniformity and in the architectural orientation of cells. Dysplasia characteristically occurs where there exists chronic irritation or inflammation, and is often found in the cervix, respiratory passages, oral cavity, and gall bladder.

Alternatively, or in addition to the presence of abnormal cell growth characterized as hyperplasia, metaplasia, or dysplasia, the presence of one or more characteristics of a transformed or malignant phenotype displayed either in vivo or in vitro within a cell sample derived from a patient, is indicative of the desirability of prophylactic/therapeutic administration of a Therapeutic that possesses the ability to modulate activity of An aforementioned protein. Characteristics of a transformed phenotype include, but are not limited to: (i) morphological changes; (ii) looser substratum attachment; (iii) loss of cell-to-cell contact inhibition; (iv) loss of anchorage dependence; (v) protease release; (vi) increased sugar transport; (vii) decreased serum requirement; (viii) expression of fetal antigens, (ix) disappearance of the 250 kDa cell-surface protein, and the like. See e.g., Richards, et al., 1986. MOLECULAR PATHOLOGY, W. B. Saunders Co., Philadelphia, Pa.

In a specific embodiment of the present invention, a patient that exhibits one or more of the following predisposing factors for malignancy is treated by administration of an effective amount of a Therapeutic: (i) a chromosomal translocation associated with a malignancy (e.g., the Philadelphia chromosome (bcr/abl) for chronic myelogenous leukemia and t(14;20) for follicular lymphoma, etc.); (ii) familial polyposis or Gardner's syndrome (possible forerunners of colon cancer); (iii) monoclonal gammopathy of undetermined significance (a possible precursor of multiple myeloma) and (iv) a first degree kinship with persons having a cancer or pre-cancerous disease showing a Mendelian (genetic) inheritance pattern (e.g., familial polyposis of the colon, Gardner's syndrome, hereditary exostosis, polyendocrine adenomatosis, Peutz-Jeghers syndrome, neurofibromatosis of Von Recklinghausen, medullary thyroid carcinoma with amyloid production and pheochromocytoma, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia and Bloom's syndrome).

In another embodiment, a Therapeutic of the present invention is administered to a human patient to prevent the progression to breast, colon, lung, pancreatic, or uterine cancer, or melanoma or sarcoma.

Hyperproliferative and Dysproliferative Disorders

In one embodiment of the present invention, a Therapeutic is administered in the therapeutic or prophylactic treatment of hyperproliferative or benign dysproliferative disorders. The efficacy in treating or preventing hyperproliferative diseases or disorders of a Therapeutic of the present invention may be assayed by any method known within the art. Such assays include in vitro cell proliferation assays, in vitro or in vivo assays using animal models of hyperproliferative diseases or disorders, or the like. Potentially effective Therapeutics may, for example, promote cell proliferation in culture or cause growth or cell proliferation in animal models in comparison to controls.

Specific embodiments of the present invention are directed to the treatment or prevention of cirrhosis of the liver (a condition in which scarring has overtaken normal liver regeneration processes); treatment of keloid (hypertrophic scar) formation causing disfiguring of the skin in which the scarring process interferes with normal renewal; psoriasis (a common skin condition characterized by excessive proliferation of the skin and delay in proper cell fate determination); benign tumors; fibrocystic conditions and tissue hypertrophy (e.g., benign prostatic hypertrophy).

Neurodegenerative Disorders

Some a FCTRX proteins are found in cell types have been implicated in the deregulation of cellular maturation and apoptosis, which are both characteristic of neurodegenerative disease. Accordingly, Therapeutics of the invention, particularly but not limited to those that modulate (or supply) activity of an aforementioned protein, may be effective in treating or preventing neurodegenerative disease. Therapeutics of the present invention that modulate the activity of an aforementioned protein involved in neurodegenerative disorders can be assayed by any method known in the art for efficacy in treating or preventing such neurodegenerative diseases and disorders. Such assays include in vitro assays for regulated cell maturation or inhibition of apoptosis or in vivo assays using animal models of neurodegenerative diseases or disorders, or any of the assays described below. Potentially effective Therapeutics, for example but not by way of limitation, promote regulated cell maturation and prevent cell apoptosis in culture, or reduce neurodegeneration in animal models in comparison to controls.

Once a neurodegenerative disease or disorder has been shown to be amenable to treatment by modulation activity, that neurodegenerative disease or disorder can be treated or prevented by administration of a Therapeutic that modulates activity. Such diseases include all degenerative disorders involved with aging, especially osteoarthritis and neurodegenerative disorders.

Disorders Related to Organ Transplantation

Some FCTRX proteins can be associated with disorders related to organ transplantation, in particular but not limited to organ rejection. Therapeutics of the invention, particularly those that modulate (or supply) activity, may be effective in treating or preventing diseases or disorders related to organ transplantation. Therapeutics of the invention (particularly Therapeutics that modulate the levels or activity of an aforementioned protein) can be assayed by any method known in the art for efficacy in treating or preventing such diseases and disorders related to organ transplantation. Such assays include in vitro assays for using cell culture models as described below, or in vivo assays using animal models of diseases and disorders related to organ transplantation, see e.g., below. Potentially effective Therapeutics, for example but not by way of limitation, reduce immune rejection responses in animal models in comparison to controls.

Accordingly, once diseases and disorders related to organ transplantation are shown to be amenable to treatment by modulation of activity, such diseases or disorders can be treated or prevented by administration of a Therapeutic that modulates activity.

Cardiovascular Disease

Proteins related to FCTRX proteins have been implicated in cardiovascular disorders, including in atherosclerotic plaque formation. Diseases such as cardiovascular disease, including cerebral thrombosis or hemorrhage, ischemic heart or renal disease, peripheral vascular disease, or thrombosis of other major vessel, and other diseases, including diabetes mellitus, hypertension, hypothyroidism, cholesterol ester storage disease, systemic lupus erythematosus, homocysteinemia, and familial protein or lipid processing diseases, and the like, are either directly or indirectly associated with atherosclerosis. Accordingly, Therapeutics of the invention, particularly those that modulate (or supply) activity or formation may be effective in treating or preventing atherosclerosis-associated diseases or disorders. Therapeutics of the invention (particularly Therapeutics that modulate the levels or activity) can be assayed by any method known in the art, including those described below, for efficacy in treating or preventing such diseases and disorders.

A vast array of animal and cell culture models exist for processes involved in atherosclerosis. A limited and non-exclusive list of animal models includes knockout mice for premature atherosclerosis (Kurabayashi and Yazaki, 1996, Int. Angiol. 15: 187-194), transgenic mouse models of atherosclerosis (Kappel et al., 1994, FASEB J. 8: 583-592), antisense oligonucleotide treatment of animal models (Callow, 1995, Curr. Opin. Cardiol. 10: 569-576), transgenic rabbit models for atherosclerosis (Taylor, 1997, Ann. N.Y. Acad. Sci 811: 146-152), hypercholesterolemic animal models (Rosenfeld, 1996, Diabetes Res. Clin. Pract. 30 Suppl.: 1-11), hyperlipidemic mice (Paigen et al., 1994, Curr. Opin. Lipidol. 5: 258-264), and inhibition of lipoxygenase in animals (Sigal et al., 1994, Ann. N.Y. Acad. Sci. 714: 211-224). In addition, in vitro cell models include but are not limited to monocytes exposed to low density lipoprotein (Frostegard et al., 1996, Atherosclerosis 121: 93-103), cloned vascular smooth muscle cells (Suttles et al., 1995, Exp. Cell Res. 218: 331-338), endothelial cell-derived chemoattractant exposed T cells (Katz et al., 1994, J. Leukoc. Biol. 55: 567-573), cultured human aortic endothelial cells (Farber et al., 1992, Am. J. Physiol. 262: H1088-1085), and foam cell cultures (Libby et al., 1996, Curr Opin Lipidol 7: 330-335). Potentially effective Therapeutics, for example but not by way of limitation, reduce foam cell formation in cell culture models, or reduce atherosclerotic plaque formation in hypercholesterolemic mouse models of atherosclerosis in comparison to controls.

Accordingly, once an atherosclerosis-associated disease or disorder has been shown to be amenable to treatment by modulation of activity or formation, that disease or disorder can be treated or prevented by administration of a Therapeutic that modulates activity.

Cytokine and Cell Proliferation/Differentiation Activity

A FCTRX protein or a cognate Therapeutic of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK.

The activity of a protein of the invention may, among other means, be measured by the following methods: Assays for T-cell or thymocyte proliferation include without limitation those described in: CURRENT PROTOCOLS IN IMMUNOLOGY, Ed by Coligan et al., Greene Publishing Associates and Wiley-Interscience (Chapter 3 and Chapter 7); Takai et al, J Immunol 137: 3494-3500, 1986; Bertagnoili et al., J Immunol 145: 1706-1712, 1990; Bertagnolli et al., Cell Immunol 133: 327-341, 1991; Bertagnolli, et al., J Immunol 149: 3778-3783, 1992; Bowman et al., J Immunol 152: 1756-1761, 1994.

Assays for cytokine production and/or proliferation of spleen cells, lymph node cells or thymocytes include, without limitation, those described by Kruisbeek and Shevach, In: CURRENT PROTOCOLS IN IMMUNOLOGY. Coligan et al., eds. Vol 1, pp. 3.12.1-14, John Wiley and Sons, Toronto 1994; and by Schreiber, In: CURRENT PROTOCOLS IN IMMUNOLOGY. Coligan eds. Vol 1 pp. 6.8.1-8, John Wiley and Sons, Toronto 1994.

Assays for proliferation and differentiation of hematopoietic and lymphopoietic cells include, without limitation, those described by Bottomly et al., In: CURRENT PROTOCOLS IN IMMUNOLOGY. Coligan et al., eds. Vol 1 pp. 6.3.1-6.3.12, John Wiley and Sons, Toronto 1991; deVries et al., J Exp Med 173: 1205-1211, 1991; Moreau et al., Nature 336: 690-692, 1988; Greenberger et al., Proc Natl Acad Sci U.S.A. 80: 2931-2938, 1983; Nordan, In: CURRENT PROTOCOLS IN IMMUNOLOGY. Coligan et al., eds. Vol 1 pp. 6.6.1-5, John Wiley and Sons, Toronto 1991; Smith et al., Proc Natl Acad Sci U.S.A. 83: 1857-1861, 1986; Measurement of human Interleukin 11-Bennett, et al. In: CURRENT PROTOCOLS IN IMMUNOLOGY. Coligan et al., eds. Vol 1 pp. 6.15.1 John Wiley and Sons, Toronto 1991; Ciarletta, et al., In: CURRENT PROTOCOLS IN IMMUNOLOGY. Coligan et al., eds. Vol 1 pp. 6.13.1, John Wiley and Sons, Toronto 1991.

Assays for T-cell clone responses to antigens (which will identify, among others, proteins that affect APC-T cell interactions as well as direct T-cell effects by measuring proliferation and cytokine production) include, without limitation, those described In: CURRENT PROTOCOLS IN IMMUNOLOGY. Coligan et al., eds., Greene Publishing Associates and Wiley-Interscience (Chapter 3Chapter 6, Chapter 7); Weinberger et al., Proc Natl Acad Sci USA 77: 6091-6095, 1980; Weinberger et al., Eur J Immun 11: 405-411, 1981; Takai et al., J Immunol 137: 3494-3500, 1986; Takai et al., J Immunol 140: 508-512, 1988.

Immune Stimulating or Suppressing Activity

A FCTRX protein or a cognate Therapeutic of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by vital (e.g., HIV) as well as bacterial or fungal infections, or may result from autoimmune disorders. More specifically, infectious diseases causes by vital, bacterial, fungal or other infection may be treatable using a protein of the present invention, including infections by HIV, hepatitis viruses, herpes viruses, mycobacteria, Leishmania species., malaria species. and various fungal infections such as candidiasis. Of course, in this regard, a protein of the present invention may also be useful where a boost to the immune system generally may be desirable, i.e., in the treatment of cancer.

Autoimmune disorders which may be treated using a protein or a cognate Therapeutic of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitus, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using a protein or a cognate Therapeutic of the invention it may also be possible to modulate immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or energy in T cells, is distinguishable from immunosuppression in that it is generally antigen-specific and persists after exposure to the tolerizing agent has ceased. Operationally, tolerance can be demonstrated by the lack of a T cell response upon re-exposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to energize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257: 789-792 (1992) and Turka et al., Proc Natl Acad Sci USA, 89: 11102-11105 (1992). In addition, murine models of GVHD (see Paul ed., FUNDAMENTAL IMMUNOLOGY, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and auto-antibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of auto-antibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythematosis in MRL/lpr/lpr mice or NZB hybrid mice, murine autoimmune collagen arthritis, diabetes mellitus in NOD mice and BB rats, and murine experimental myasthenia gravis (see Paul ed., FUNDAMENTAL IMMUNOLOGY, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic vital diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-vital immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class II a chain protein and an MHC class II β chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein or a cognate Therapeutic of the invention may, among other means, be measured by the following methods: Suitable assays for thymocyte or splenocyte cytotoxicity include, without limitation, those described In: CURRENT PROTOCOLS IN IMMUNOLOGY. Coligan et al., eds. Greene Publishing Associates and Wiley-Interscience (Chapter 3, Chapter 7); Herrmann et al., Proc Natl Acad Sci USA 78: 2488-2492, 1981; Herrmann et al., J Immunol 128: 1968-1974, 1982; Handa et al., J Immunol 20: 1564-1572, 1985; Takai et al., J Immunol 137: 3494-3500, 1986; Takai et al., J Immunol 140: 508-512, 1988; Herrmann et al., Proc Natl Acad Sci USA 78: 2488-2492, 1981; Herrmann et al., J Immunol 128: 1968-1974, 1982; Handa et al., J Immunol 18: 1564-1572, 1985; Takai et al, J Immunol 137: 3494-3500, 1986; Bowman et al., J Virology 61: 1992-1998; Takai et al., J Immunol 140: 508-512, 1988; Bertagnolli et al., Cell Immunol 133: 327-341, 1991; Brown et al., J Immunol 153: 3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J Immunol 144: 3028-3033, 1990; and Mond and Brunswick In: CURRENT PROTOCOLS IN IMMUNOLOGY. Coligan et al., (eds.) Vol 1 pp. 3.8.1-3.8.16, John Wiley and Sons, Toronto 1994.

Mixed lymphocyte reaction (MLR) assays (which will identify, among others, proteins that generate predominantly Th1 and CTL responses) include, without limitation, those described In: CURRENT PROTOCOLS IN IMMUNOLOGY. Coligan et al., eds. Greene Publishing Associates and Wiley-Interscience (Chapter 3, Chapter 7); Takai et al., J Immunol 137: 3494-3500, 1986; Takai et al., J Immunol 140: 508-512, 1988; Bertagnolli et al., J Immunol 149: 3778-3783, 1992.

Dendritic cell-dependent assays (which will identify, among others, proteins expressed by dendritic cells that activate naive T-cells) include, without limitation, those described in: Guery et al., J Immunol 134: 536-544, 1995; Inaba et al., JExp Med 173: 549-559, 1991; Macatonia et al., J Immunol 154: 5071-5079, 1995; Porgador et al., J Exp Med 182: 255-260, 1995; Nair et al., J Virol 67: 4062-4069, 1993; Huang et al., Science 264: 961-965, 1994; Macatonia et al., J Exp Med 169: 1255-1264, 1989; Bhardwaj et al., J Clin Investig 94: 797-807, 1994; and Inaba et al., J Exp Med 172: 631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13: 795-808, 1992; Gorczyca et al., Leukemia 7: 659-670, 1993; Gorczyca et al., Cancer Res 53: 1945-1951, 1993; Itoh et al., Cell 66: 233-243, 1991; Zacharchuk, J Immunol 145: 4037-4045, 1990; Zamai et al., Cytometry 14: 891-897, 1993; Gorczyca et al., Internat J Oncol 1: 639-648, 1992.

Assays for proteins that influence early steps of T-cell commitment and development include, without limitation, those described in: Antica et al., Blood 84: 111-117, 1994; Fine et al., Cell Immunol 155: 111-122, 1994; Galy et al., Blood 85: 2770-2778, 1995; Toki et al., Proc Nat Acad Sci USA 88: 7548-7551, 1991.

Hematopoiesis Regulating Activity

A FCTRX protein or a cognate Therapeutic of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15: 141-151, 1995; Keller et al., Mol. Cell. Biol. 13: 473-486, 1993; McClanahan et al., Blood 81: 2903-2915, 1993.

Assays for stem cell survival and differentiation (which will identify, among others, proteins that regulate lympho-hematopoiesis) include, without limitation, those described in: Methylcellulose colony forming assays, Freshney, In: CULTURE OF HEMATOPOIETIC CELLS. Freshney, et al. (eds.) Vol pp. 265-268, Wiley-Liss, Inc., New York, N.Y 1994; Hirayama et al., Proc Natl Acad Sci USA 89: 5907-5911, 1992; McNiece and Briddeli, In: CULTURE OF HEMATOPOIETIC CELLS. Freshney, et al. (eds.) Vol pp. 23-39, Wiley-Liss, Inc., New York, N.Y. 1994; Neben et al., Exp Hematol 22: 353-359, 1994; Ploemacher, In: CULTURE OF HEMATOPOIETIC CELLS. Freshney, et al. eds. Vol pp. 1-21, Wiley-Liss, Inc., New York, N.Y. 1994; Spooncer et al., In: CULTURE OF HEMATOPOIETIC CELLS. Freshhey, et al., (eds.) Vol pp. 163-179, Wiley-Liss, Inc., New York, N.Y. 1994; Sutherland, In: CULTURE OF HEMATOPOIETIC CELLS. Freshney, et al., (eds.) Vol pp. 139-162, Wiley-Liss, Inc., New York, N.Y. 1994.

Tissue Growth Activity

A FCTRX protein or a cognate Therapeutic of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein or a cognate Therapeutic of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein or a cognate Therapeutic of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendonitis, carpal tunnel syndrome and other tendon or ligament defects. The compositions may also include an appropriate matrix and/or sequestering agent as a career as is well known in the art.

A protein or a cognate Therapeutic of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, EPIDERMAL WOUND HEALING, pp. 71-112 (Maibach and Rovee, eds.), Year Book Medical Publishers, Inc., Chicago, as modified by Eaglstein and Menz, J. Invest. Dermatol 71: 382-84 (1978).

Activin/Inhibin Activity

A FCTRX protein or a cognate Therapeutic of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin a family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-b group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91: 562-572, 1972; Ling et al., Nature 321: 779-782, 1986; Vale et al., Nature 321: 776-779, 1986; Mason et al., Nature 318: 659-663, 1985; Forage et al., Proc Natl Acad Sci USA 83: 3091-3095, 1986.

Chemotactic/Chemokinetic Activity

A protein or a cognate Therapeutic of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: CURRENT PROTOCOLS IN IMMUNOLOGY, Coligan et al., eds. (Chapter 6.12, MEASUREMENT OF ALPHA AND BETA CHEMOKINES 6.12.1-6.12.28); Taub et al. J Clin Invest 95: 1370-1376, 1995; Lind et al. APMIS 103: 140-146, 1995; Muller et al., Eur J Immunol 25: 1744-1748; Gruber et al. J Immunol 152: 5860-5867, 1994; Johnston et al., J Immunol 153: 1762-1768, 1994.

Hemostatic and Thrombolytic Activity

A protein or a cognate Therapeutic of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolytic activity include, without limitation, those described in: Linet et al., J. Clin. Pharmacol. 26: 131-140, 1986; Burdick et al., Thrombosis Res. 45: 413-419, 1987; Humphrey et al., Fibrinolysis 5: 71-79 (1991); Schaub, Prostaglandins 35: 467-474, 1988.

Receptor/Ligand Activity

A protein or a cognate Therapeutic of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell—cell interactions and their ligands (including without limitation, cellular adhesion molecules (such as selectins, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: CURRENT PROTOCOLS IN IMMUNOLOGY, Ed by Coligan, et al., Greene Publishing Associates and Wiley-Interscience (Chapter 7.28, Measurement of Cellular Adhesion under static conditions 7.28.1-7.28.22), Takai et al., Proc Natl Acad Sci USA 84: 6864-6868, 1987; Bierer et al., J. Exp. Med. 168: 1145-1156, 1988; Rosenstein et al., J. Exp. Med. 169: 149-160 1989; Stoltenborg et al., J Immunol Methods 175: 59-68, 1994; Stitt et al., Cell 80: 661-670, 1995.

Anti-Inflammatory Activity

Proteins or cognate Therapeutics of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

Other Activities

A protein or a cognate Therapeutic of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Neural disorders in general include Parkinson's disease, Alzheimer's disease, Huntington's disease, multiple sclerosis, amyotrophic lateral sclerosis (ALS), peripheral neuropathy, tumors of the nervous system, exposure to neurotoxins, acute brain injury, peripheral nerve trauma or injury, and other neuropathies, epilepsy, and/or tremors.

The invention will be further illustrated in the following non-limiting examples.

### EXAMPLES

### Example 1

### Molecular Cloning of a Mature Form (30664188.0.m99) Polypeptide from Cline 30664188.0.99

A mature form of clone 30664188.0.99, coding for residues 24 to 370 of the amino acid sequence of SEQ ID NO:2, was cloned. This fragment was designated 30664188.0.m99 and corresponds to the polypeptide sequence remaining after a signal peptide predicted to be cleaved between residues 23 and 24 has been removed. The following oligonucleotide primers were designed to PCR amplify the predicted mature form of 30664188.0.99.

| | |
|---|---|
| 30664188 Eco Forward: | |
| CTCGTC GAATTC ACC CCG CAG AGC | (SEQ ID NO:5) |
| GCA TCC ATC AAA GC | |
| 3066418 Xho Reverse: | |
| CTCGTC CTC GAG TCG AGG TGG | (SEQ ID NO:6) |
| TCT TGA GCT GCA GAT ACA | |

The forward primer included an in frame EcoRI restriction site, and the reverse primer included an XhoI restriction site. The EcoRI/XhoI fragment is compatible with the pET28a E. coli expression vector and with the pMelV5His baculovirus expression vector.

PCR reactions were set up using 5 ng human spleen and fetal lung cDNA templates. The reaction mixtures contained 1 microM of each of the 30664188 Eco Forward and 3066418 Xho Reverse primers, 5 micromoles dNTP (Clontech Laboratories, Palo Alto Calif.) and 1 microliter of 50×Advantage-HF 2 polymerase (Clontech Laboratories, Palo Alto Calif.) in 50 microliter volume. The following reaction conditions were used:
a) 96° C. 3 minutes
b) 96° C. 30 seconds denaturation
c) 70° C. 30 seconds, primer annealing. This temperature was gradually decreased by 1° C./cycle
d) 72° C. 1 minute extension.
Repeat steps b-d 10 times
e) 96° C. 30 seconds denaturation
f) 60° C. 30 seconds annealing
g) 72° C. 1 minute extension
Repeat steps e-g 25 times
h) 72° C. 5 minutes final extension

The amplified product expected to have 1041 bp was detected by agarose gel electrophoresis in both samples. The fragments were purified from agarose gel and ligated to pCR2.1 vector (Invitrogen, Carlsbad, Calif.). The cloned inserts were sequenced using M13 Forward, M13 Reverse and the following gene specific primers:

| | |
|---|---|
| 3066418 S1: | |
| GGA CGA TGG TGT GGA CAC AAG, | (SEQ ID NO:7) |
| 3066418 S2: | |
| CTT GTG TCC ACA CCA TCG TCC, | (SEQ ID NO:8) |
| 3066418 S3: | |
| TAT CGA GGC AGG TCA TAC CAT | (SEQ ID NO:9) |
| and | |
| 3066418 S4: | |
| ATG GTA TGA CCT GCC TCG ATA. | (SEQ ID NO:10) |

The cloned inserts were verified as an open reading frame coding for the predicted mature form of 30664188.0.99. The construct derived from fetal lung, called 30664188-S311a, was used for further subcloning into expression vectors (see below). The nucleotide sequence of 30664188-S11a within the restriction sites was found to be 100% identical to the corresponding fragment in the ORF of 30664188.0.99 (Table. 1; SEQ ID NO:1).

### Example 2

### Preparation of Mammalian Expression Vector pCEP4/Sec

FCTRX nucleic acids were expressed in mammalian cells in a vector named pCEP4/SEC. The vector was prepared using the oligonucleotide primers,
pSec-V5-His Forward
CTCGTCCTCGAGGGTAAGCCTATCCCTAAC (SEQ ID NO:11) and
pSec-V5-His Reverse
CTCGTCGGGCCCCTGATCAGCGGGTTTAAAC (SEQ ID NO:12),

These primers were designed to amplify a fragment from the pcDNA3.1-V5His (Invitrogen, Carlsbad, Calif.) expression vector that includes V5 and His6. The PCR product was digested with XhoI and ApaI and ligated into the XhoI/ApaI digested pSecTag2 B vector harboring an Ig kappa leader sequence (Invitrogen, Carlsbad Calif.). The correct structure of the resulting vector, pSecV5His, including an in-frame Ig-kappa leader and V5-His6 was verified by DNA sequence analysis. The vector pSecV5His was digested with PmeI and NheI to provide a fragment retaining the above elements in the correct frame. The PmeI-NheI fragment was ligated into the BamHI/Klenow and NheI treated vector pCEP4 (Invitrogen, Carlsbad, Calif.). The resulting vector was named pCEP4/Sec and includes an in-frame Ig kappa leader, a site for insertion of a clone of interest, V5 and 6×His under control of the PCMV and/or the PT7 promoter. pCEP4/Sec is an expression vector that allows heterologous protein expression and secretion by fusing any protein to the Ig Kappa chain signal peptide. Detection and purification of the expressed protein are aided by the presence of the V5 epitope tag and 6×His tag at the C-terminus (Invitrogen, Carlsbad, Calif.).

### Example 3

### Expression of 30664188.m99 Polypeptide in E. coli

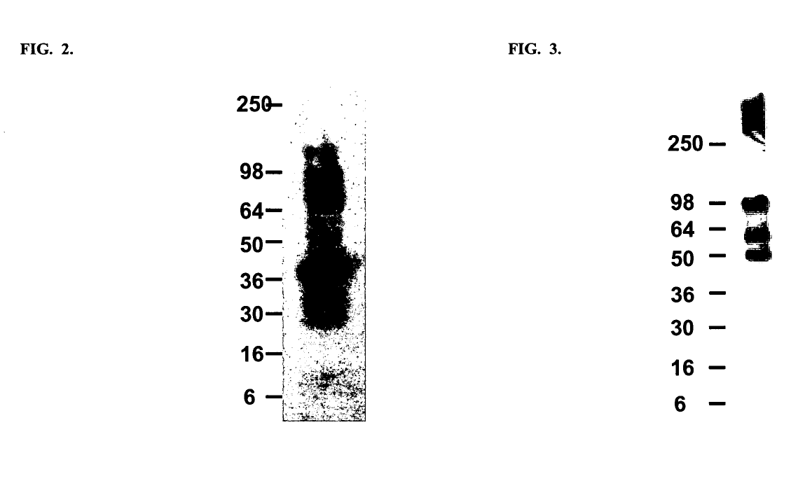

The vector pRSETA (InVitrogen Inc., Carlsbad, Calif.) was digested with XhoI and NcoI restriction enzymes. Oligonucleotide linkers
CATGGTCAGCCTAC (SEQ ID NO:13); and
TCGAGTAGGCTGAC (SEQ ID NO:14)
   were annealed at 37 degrees Celsius and ligated into the XhoI-NcoI treated pRSETA. The resulting vector was confirmed by restriction analysis and sequencing and was named pETMY. The BamHI-XhoI fragment containing the 30664188 sequence (Example 3) was ligated into BamHI-XhoI digested pETMY. The resulting expression vector was named pETMY-30664188. In this vector, 30664188 is fused to the T7 epitope and a 6×His tag at its N-terminus The plasmid pETMY-30664188 was then transfected into the E. coli expression host BL21(DE3, pLys) (Novagen, Madison, Wis.) and expression of the protein was induced according to the manufacturer's instructions. After induction, the E. coli cells were harvested, and proteins were analyzed by Western blotting using anti-His6Gly antibody (Invitrogen, Carlsbad, Calif.). FIG. 2 shows 30664188.m99 was expressed as a protein of apparent molecular weight 40 kDa. This approximates the molecular weight expected for the 30664188.m99 sequence.

### Example 4

### Expression of 30664188.m99 Polypeptide in Human Embryonic Kidney 293 Cells

The EcoRI-XhoI fragment containing the 30664188.m99 sequence was isolated from 30664188-S311a (Example 1) and subcloned into the vector pE28a (Novagen, Madison, Wis.) to give the plasmid pET28a-30664188. Subsequently, pET28a-30664188 was partially digested with BamHI restriction enzyme, and then completely digested with XhoI. A fragment of 1.1 kb was isolated and ligated into BamHI-XhoI digested pCEP4/Sec (Example 2) to generate expression vector pCEP4/Sec-30664188. The pCEP4/Sec-30664188 vector was transfected into human embryonic kidney 293 cells (ATCC No. CRL-1573, Manassas, Va.) using the LipofectaminePlus reagent following the manufacturer's instructions (Gibco/BRL/Life Technologies, Rockville, Md.). The cell pellet and supernatant were harvested 72 hours after transfection and examined for expression of the 30664188.m99 protein by Western blotting of an SDS-PAGE run under reducing conditions using an anti-V5 antibody. FIG. 3 shows that 30664188.m99 is expressed as three discrete protein bands of apparent molecular weight 50, 60, and 98 kDa secreted by 293 cells. The 50 kDa band migrated at a sized expected for a monomer glycosylated form of 30664188.m99, and the 98 kDa band migrated at a sized consistent with a dimer of the monomer form.

### Example 5

### Radiation Hybrid Mapping of 30664188.0.99.

Radiation hybrid mapping using human chromosome markers was carried out for clone 30664188.0.99. The procedure used to obtain these results is analogous to that described in Steen, R G et al. (A High-Density Integrated Genetic Linkage and Radiation Hybrid Map of the Laboratory Rat, Genome Research 1999 (Published Online on May 21, 1999) Vol. 9, AP1-AP8, 1999). A panel of 93 cell clones containing the randomized radiation-induced human chromosomal fragments was screened in 96 well plates using PCR primers designed to identify the sought clones in a unique fashion. Clone 30664188.0.99 was found to be located on chromosome 11, 3.1 cR from marker WI-9345 and 1.7 cR from marker CHLC.GATA6C11.

### Example 6

### Expression and Purification of 30664188.m99 Protein

The segment representing the mature protein cloned in Example 1 was excised and subcloned into the vector pCEP4/Sec (Example 2) suitable for transfection of HEK 293 cells under the control of the pCEP4 promoter. The resulting vector was named pCEP4/Sec/30664188.

HEK 293 cells were grown in Dulbecco's modified eagle's medium (DMEM)/10% fetal bovine serum medium to 90% confluence. The cells were transfected with pCEP4sec or pCEP4sec/30664188.m99 using Lipofectamine 2000 according to the manufacturer's specifications (Gibco/BRL/Life Technologies, Rockville, Md.). Transfected cells were incubated for 2 days with DMEM and conditioned medium was prepared by collection of cell supernatants. The conditioned medium was enriched by Talon metal affinity chromatography (Clontech, Palo Alto, Calif.). Briefly, 7 ml of conditioned medium was incubated with 1 ml of Talon metal affinity resin in spin columns. The spin columns were washed twice with one ml of PBS. The columns were then eluted twice with 0.65 ml of PBS/0.5M imidazole pH 8.0 and the eluates pooled. Imidazole was removed by buffer exchange dialysis into PBS using Microcon centrifugal filter devices (Millipore Corp., Bedford, Mass.). The enriched gene products were stored at 4° C.

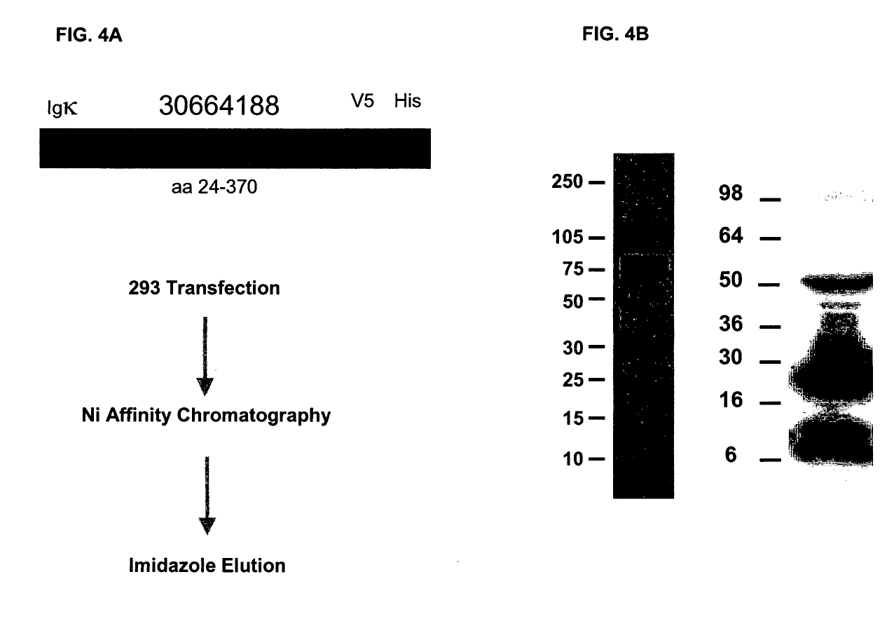

The purified protein obtained was subjected to SDS-PAGE under reducing conditions and probed with an anti-V5 antibody, which was detected with an enzyme label. The results of two separate transfection and purification runs are shown in the gels. They show that the product is a mixture of V5-containing polypeptides. The largest has an apparent molecular weight of about 50 kDa (FIG. 4). The program ProSite predicts one N-glycosylation site in the mature protein. Glycosylation may explain the apparent molecular weight found. Thus the 50 kDa band is consistent with the length expected for full length gene product. Other bands, preponderantly having apparent molecular weights of about 20-25 kDa also arise. These are presumed to be the result of proteolysis occurring either intracellularly within the 293 cells or extracellularly after secretion from them.

### Example 7

### Real Time Tissue Expression Profiling of Sequence 30664188 by Quantitative PCR

Real time PCR was followed for multiple tissue or cell samples by monitoring release of a 5′ fluorogenic label from a specific oligonucleotide probe bearing a 3′ quencher. The target sequence specific for the 30664188 transcript is detected and monitored in real time, as the PCR takes place using the fluorogenic 5′ nuclease assay performed with the TaqMan® PCR Reagent Kit (Roche Molecular Systems, Inc.) and the Perkin-Elmer Biosystems ABI PRISM® 7700 Sequence Detection System.

Probes and primers were designed according to Perkin Elmer Biosystem's Primer Express Software package (version I for Apple Computer's Macintosh Power PC) using the sequence of 30664188 as input. Default settings were used for reaction conditions and the following parameters were set before selecting primers: primer concentration=250 nM, primer melting temperature (Tₘ) range=58°-60° C., primer optimal Tₘ=59° C., maximum primer difference=2° C., probe does not have 5′ G, probe Tₘ must be 10° C. greater than primer Tₘ, applicon size 75 bp to 100 bp. Three sets of primers and probe were synthesized by Synthegen (Houston, TX, USA), and were HPLC purified twice to remove uncoupled dye. Mass spectroscopy was used to verify efficient coupling of reporter and quencher dyes to the 5′ and 3′ ends of the probe, respectively.

PCR preparation and conditions included the following steps: Sample RNA from each tissue (poly A+ RNA, 2.8 pg) and the cell lines (total RNA, 70 ng) was spotted in each well of a 96 well PCR plate (Perkin Elmer Biosystems). A panel of 41 normal human tissues and 55 human cancer cell lines was employed (see Table 6).

TABLE 3
Results of Real Time TaqMan ™ Tissue Profiling

| | | Relative Expression (%) | | |
|---|---|---|---|---|
| | Normal & Tumor Tissues | Ag33 | Ag66 | Ag168 |
| 1 | Endothelial cells | 1.66 | 1.23 | 0.00 |
| 2 | Endothelial cells (treated) | 2.80 | 1.51 | 0.00 |
| 3 | Pancreas | 36.35 | 28.72 | 37.89 |
| 4 | Pancreatic ca. CAPAN 2 | 1.05 | 0.46 | 0.00 |
| 5 | Adipose | 10.37 | 30.57 | 54.34 |
| 6 | Adrenal gland | 100.00 | 100.00 | 0.00 |
| 7 | Thyroid | 20.45 | 8.19 | 1.42 |
| 8 | Salivary gland | 6.52 | 6.75 | 0.19 |
| 9 | Pituitary gland | 5.83 | 4.01 | 0.00 |
| 10 | Brain (fetal) | 2.16 | 2.32 | 0.00 |
| 11 | Brain (whole) | 3.54 | 2.66 | 0.00 |
| 12 | Brain (amygdala) | 1.29 | 0.85 | 0.05 |
| 13 | Brain (cerebellum) | 1.30 | 1.02 | 0.00 |
| 14 | Brain (hippocampus) | 3.26 | 1.88 | 0.00 |
| 15 | Brain (hypothalamus) | 42.93 | 37.11 | 46.98 |
| 16 | Brain (substantia nigra) | 2.05 | 0.00 | 0.00 |
| 17 | Brain (thalamus) | 0.39 | 0.25 | 0.00 |
| 18 | Spinal cord | 4.58 | 2.78 | 0.00 |
| 19 | CNS ca. (glio/astro) U87-MG | 0.00 | 0.00 | 0.00 |
| 20 | CNS ca. (glio/astro) U-118-MG | 0.00 | 0.07 | 0.00 |
| 21 | CNS ca. (astro) SW1783 | 1.94 | 1.49 | 0.00 |
| 22 | CNS ca.* (neuro; met) SK-N-AS | 2.05 | 1.04 | 0.00 |
| 23 | CNS ca. (astro) SF-539 | 0.32 | 0.13 | 0.00 |
| 24 | CNS ca. (astro) SNB-75 | 5.29 | 5.26 | 0.00 |
| 25 | CNS ca. (glio) SNB-19 | 3.85 | 3.64 | 0.03 |
| 26 | CNS ca. (glio) U251 | 2.82 | 1.67 | 0.00 |
| 27 | CNS ca. (glio) SF-295 | 82.36 | 53.59 | 100.00 |
| 28 | Heart | 14.66 | 13.58 | 1.42 |
| 29 | Skeletal muscle | 1.29 | 0.96 | 0.00 |
| 30 | Bone marrow | 1.23 | 0.69 | 0.00 |
| 31 | Thymus | 6.04 | 2.78 | 0.00 |
| 32 | Spleen | 2.24 | 1.78 | 0.00 |
| 33 | Lymph node | 5.79 | 3.74 | 0.03 |
| 34 | Colon (ascending) | 2.06 | 3.61 | 0.01 |
| 35 | Stomach | 24.66 | 26.06 | 15.07 |
| 36 | Small intestine | 5.95 | 5.11 | 0.02 |
| 37 | Colon ca. SW480 | 0.00 | 0.00 | 0.00 |
| 38 | Colon ca.* (SW480 met)SW620 | 0.00 | 0.00 | 0.00 |
| 39 | Colon ca. HT29 | 0.00 | 0.02 | 0.00 |
| 40 | Colon ca. HCT-116 | 0.00 | 0.00 | 0.00 |
| 41 | Colon ca. CaCo-2 | 0.01 | 0.03 | 0.00 |
| 42 | Colon ca. HCT-15 | 0.00 | 0.00 | 0.00 |
| 43 | Colon ca. HCC-2998 | 0.00 | 0.00 | 0.00 |
| 44 | Gastric ca.* (liver met) NCI-N87 | 0.00 | 0.00 | 0.00 |
| 45 | Bladder | 2.92 | 13.21 | 0.00 |
| 46 | Trachea | 24.49 | 15.82 | 17.43 |
| 47 | Kidney | 5.40 | 4.09 | 0.23 |
| 48 | Kidney (fetal) | 14.16 | 10.08 | 0.00 |
| 49 | Renal ca. 786-0 | 0.00 | 0.00 | 0.00 |
| 50 | Renal ca. A498 | 0.82 | 0.55 | 0.00 |
| 51 | Renal ca. RXF 393 | 0.08 | 0.06 | 0.00 |
| 52 | Renal ca. ACHN | 0.69 | 0.44 | 0.00 |
| 53 | Renal ca. UO-31 | 0.12 | 0.09 | 0.00 |
| 54 | Renal ca. TK-10 | 1.50 | 0.57 | 0.00 |
| 55 | Liver | 5.37 | 4.45 | 1.75 |
| 56 | Liver (fetal) | 1.56 | 1.12 | 0.00 |
| 57 | Liver ca. (hepatoblast) HepG2 | 0.00 | 0.00 | 0.00 |
| 58 | Lung | 0.34 | 1.30 | 0.00 |
| 59 | Lung (fetal) | 2.68 | 1.62 | 0.00 |
| 60 | Lung ca. (small cell) LX-1 | 0.00 | 0.00 | 0.00 |
| 61 | Lung ca. (small cell) NCI-H69 | 0.63 | 0.44 | 0.00 |
| 62 | Lung ca. (s.cell var.) SHP-77 | 0.00 | 0.00 | 0.01 |
| 63 | Lung ca. (large cell)NCI-H460 | 0.63 | 0.48 | 0.00 |
| 64 | Lung ca. (non-sm. cell) A549 | 6.98 | 6.12 | 0.00 |
| 65 | Lung ca. (non-s.cell) NCI-H23 | 0.22 | 0.12 | 0.00 |
| 66 | Lung ca (non-s.cell) HOP-62 | 2.78 | 2.03 | 0.00 |
| 67 | Lung ca. (non-s.cl) NCI-H522 | 0.03 | 0.01 | 0.00 |
| 68 | Lung ca.(squam.) SW 900 | 11.50 | 11.19 | 2.40 |
| 69 | Lung ca. (squam.) NCI-H596 | 4.97 | 4.09 | 0.00 |
| 70 | Mammary gland | 32.76 | 31.43 | 24.32 |
| 71 | Breast ca.* (pl. effusion) MCF-7 | 0.00 | 0.00 | 0.00 |
| 72 | Breast ca.* (pl.ef) MDA-MB-231 | 0.00 | 0.01 | 0.00 |
| 73 | Breast ca.* (pl. effusion) T47D | 0.00 | 0.11 | 0.00 |
| 74 | Breast ca. BT-549 | 7.59 | 7.38 | 0.00 |
| 75 | Breast ca. MDA-N | 0.00 | 0.02 | 0.00 |
| 76 | Ovary | 9.61 | 11.03 | 0.00 |
| 77 | Ovarian ca. OVCAR-3 | 0.84 | 0.22 | 0.00 |
| 78 | Ovarian ca. OVCAR-4 | 0.31 | 0.20 | 0.00 |
| 79 | Ovarian ca. OVCAR-5 | 81.79 | 78.46 | 93.95 |
| 80 | Ovarian ca. OVCAR-8 | 2.08 | 1.54 | 0.00 |
| 81 | Ovarian ca. IGROV-1 | 3.00 | 2.05 | 0.00 |
| 82 | Ovarian ca.* (ascites) SK-OV-3 | 0.12 | 0.05 | 0.00 |
| 83 | Myometrium | 5.08 | 7.38 | 0.26 |
| 84 | Uterus | 8.30 | 4.94 | 0.20 |
| 85 | Placenta | 7.33 | 5.79 | 0.29 |
| 86 | Prostate | 5.56 | 4.01 | 0.04 |
| 87 | Prostate ca.* (bone met)PC-3 | 19.75 | 9.47 | 0.00 |
| 88 | Testis | 20.88 | 21.46 | 6.89 |
| 89 | Melanoma Hs688(A).T | 0.89 | 0.45 | 0.00 |
| 90 | Melanoma* (met) Hs688(B).T | 0.91 | 0.46 | 0.00 |
| 91 | Melanoma UACC-62 | 0.21 | 0.13 | 0.00 |
| 92 | Melanoma M14 | 0.68 | 0.20 | 0.00 |
| 93 | Melanoma LOX IMVI | 1.57 | 0.99 | 0.00 |
| 94 | Melanoma* (met) SK-MEL-5 | 1.47 | 0.50 | 0.00 |
| 95 | Melanoma SK-MEL-28 | 5.95 | 4.45 | 0.00 |
| 96 | Melanoma UACC-257 | 3.69 | 3.21 | 1.99 |

In Table 3, the following abbreviations are used:
ca. = carcinoma,
* = established from metastasis,
met = metastasis,
s cell var = small cell variant,
non-s = non-sm = non-small,
squam = squamous,
pl. eff = pl effusion = pleural effusion,
glio = glioma,
astro = astrocytoma, and
neuro = neuroblastoma.

PCR cocktails including two sets primers and probes (a 30664188-specific and a reference gene-specific probe, commonly β-actin and/or GAPDH, multiplexed with the 30664188 probe) were set up using 1×TaqMan™ PCR Master Mix for the PE Biosystems 7700, with 5 mM MgCl2, dNTPs (dA, dG, dC, dU at 1:1:1:2 ratios), 0.25 U/ml AmpliTaq Gold™ (PE Biosystems), and 0.4 U/μl RNase inhibitor, and 0.25 U/ml reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

The TaqMan probes and primers used were:

| | |
|---|---|
| Ag33 (F): | |
| 5′-CGCTTGGCATCATCATTGAG-3′, | (SEQ ID NO:15) |
| Ag33 (R): | |
| 5′-CGGTATCGAGGCAGGTCATAC-3′, | (SEQ ID NO:16) |
| and | |
| Ag33 (P): | |
| TET-5′- | (SEQ ID NO:17) |
| TCCAGGTCAACTTTTGACTTCCGGTCA- | |
| 3′-TAMRA; | |
| Ag66 (R): | |
| 5′-CACAAGGAAGTTCCTCCAAGGATA-3′, | (SEQ ID NO:18) |
| Ag66 (F): | |
| 5′-AATCCAGGTTTAGCCACAAAGTAGTC, | (SEQ ID NO:19) |
| and | |
| Ag66 (P): | |
| FAM-5′- | (SEQ ID NO:20) |
| AGAACGAACCAAATTAAAATCACATTCAAGTCCGA- | |
| TAMRA; | |
| and | |
| Ag 168 (F): | |
| 5′-GCATGTGCAGGACCTCCAGT-3′, | (SEQ ID NO:21) |
| Ag 168 (R): | |
| 5′-TCCACGTTGCCTCCTCGT-3′, | (SEQ ID NO:22) |
| and | |
| Ag 168 (P): | |
| TET-5′- | (SEQ ID NO:23) |
| CAGTTCCACAGCCACAATTTCCTCCAC- | |
| 3′-TAMRA. | |

The results are provided in Table 6. Among normal tissues examined, clone 30664188 is highly expressed in pancreas, adrenal gland, adipose tissue, stomach, trachea, mammary gland and testis. Among various cancer cell lines, the clone is strongly expressed specifically in CNS cancer (CNS ca. (glio) SF-295), lung cancer (squamous cells, SW 900) and ovarian cancer (ovarian ca. OVCAR-5).

### Example 8

### The Clone 30664188.0.m99 Protein Induces Cellular DNA Synthesis

Human CCD-1070 fibroblast cells (ATCC No. CRL-2091, Manassas, Va.) or murine NIH 3T3 (ATCC No. CRL-1658, Manassas, Va.) fibroblast cells were cultured in DMEM supplemented with 10% fetal bovine serum or 10% calf serum respectively. Fibroblasts were grown to confluence at 37° C. in 10% CO₂/air. Cells were then starved in DMEM for 24 h. pCEP4/Sec (Example 2) or pCEP4/Sec/30664188.m99 (Example 6) enriched conditioned medium was added (10 microL/100 microL of culture) for 18 h. BrdU (10 uM) was then added and incubated with the cells for 5 h. BrdU incorporation was assayed by colorimetric immunoassay according to the manufacturer's specifications (Boehringer Mannheim, Indianapolis, Ind.).

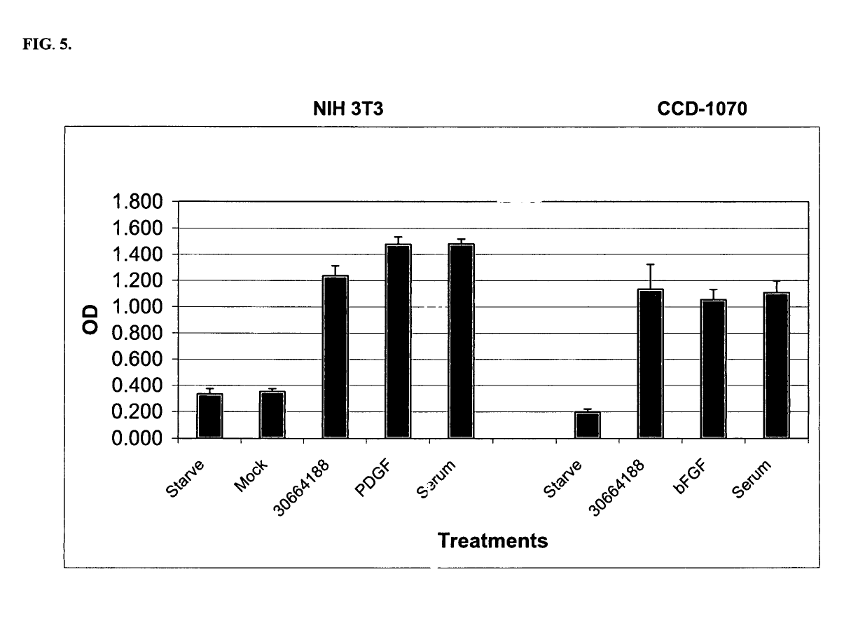

FIG. 5 demonstrates that 30664188.m99 induced an approximate four- to five-fold increase in BrdU incorporation in either cell type compared to cells treated with control conditioned medium or untreated cells. The proliferative increase observed was similar to the increase in BrdU incorporation induced by platelet derived FCTRX (PDGF), basic fibroblast growth factor (bFGF), or serum treatment. Additionally, 30664188.m99 partially purified conditioned medium did not induce BrdU incorporation in human MG-63 epithelial cells or CCD1106 keratinocytes (data not shown). These results suggest that 30664188 selectively induces DNA synthesis in human and mouse fibroblasts, but not in epithelial cell lines.

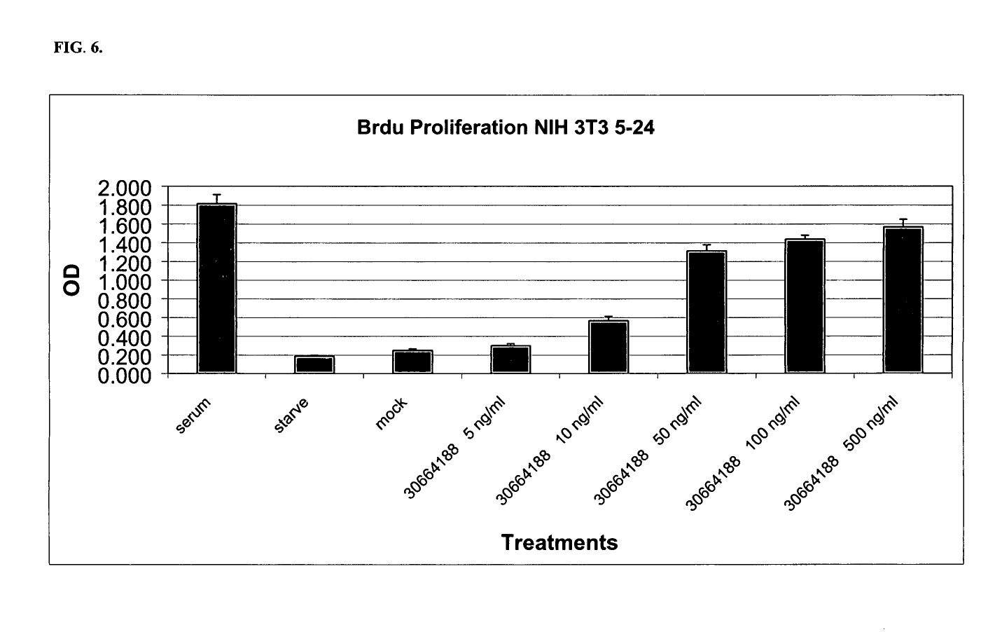

In separate experiments, CCD-1070 cells and MG-63 osteosarcoma cells (ATCC Cat. No. CRL-1427) treated with pCEP4/Sec/30664188 each incorporated BrdU in a dose-dependent fashion, with 1 ug/mL providing the full effect (approximately 2.5- to 3-fold increase over control), 100 ng/mL providing slightly less than one-half the effect, and 10 and 1 ng/mL providing approximately control levels of incorporation. Furthermore, the dose response of NIH 3T3 cells shows that a 50% response occurs between doses of 10 and 50 ng/mL of pCEP4/Sec/30664188 (FIG. 6).

### Example 9

### Induction of Proliferation of NIH 3T3 Cells by 30664188.m99

Murine NIH 3T3 fibroblasts were plated at 40% confluency and cultured in DMEM supplemented with 10% fetal bovine serum or 10% calf serum for 24 hrs. The culture medium was removed and replaced with an equivalent volume of pCEP4/Sec (Example 2) or pCEP4/Sec/30664188 (Example 6) conditioned medium. After 48 h, cells were photographed with a Zeiss Axiovert 100. Cell numbers were determined by trypsinization followed by counting using a Coulter Z1 Particle Counter.

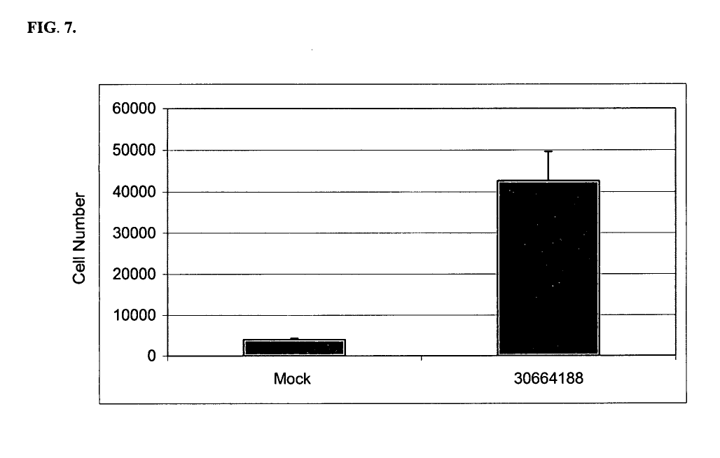

Treatment of NIH 3T3 fibroblasts with conditioned medium from 30664188 transfected HEK293 kidney epithelial cells resulted in a 6 to 8 fold increase in cell number over a two day period (FIG. 7). Cells treated with control conditioned medium from HEK293 cells transfected with the pCEP4/Sec vector alone demonstrated little or no growth (FIG. 7).

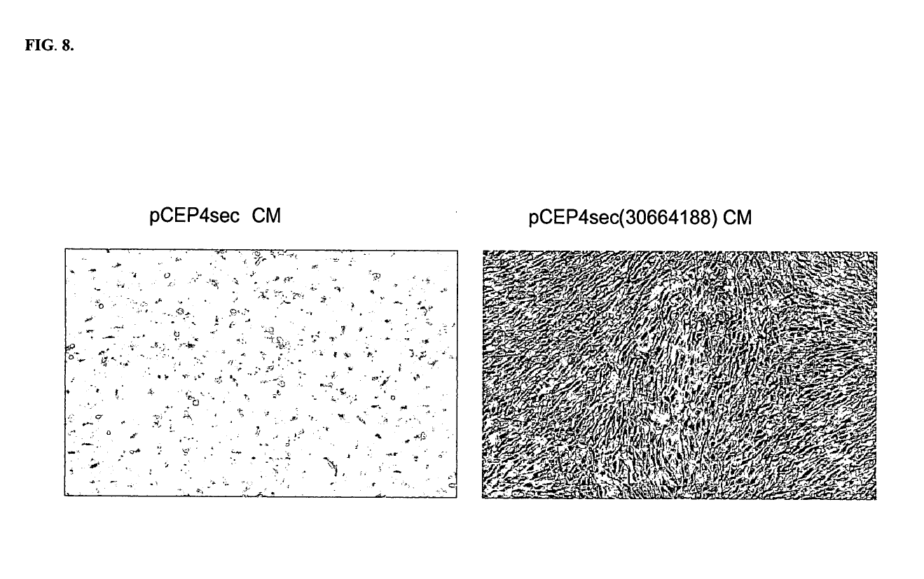

To determine whether 30664188.m99 conditioned medium was able to induce phenotypic changes characteristic of cellular transformation, cells treated with either 30664188 conditioned medium or mock conditioned medium were examined by light microscopy. FIG. 8 shows that NIH 3T3 cells treated with 30664188.m99, but not control treated NIH 3T3 cells, showed a marked increase in cell number, as well as refractile properties. Loss of contact inhibition of growth was evident. The cobblestone appearance characteristic of confluent NIH 3T3 cells was lost and density independent growth was evident. The latter was also suggested by the more rounded appearance of the NIH 3T3 cells due to subtle retraction. Transfection of pCEP4/Sec/30664188.m99 also showed nearly identical potency in transformation potential 2 to 5 days in culture. After 7 to 10 days in culture, however, the morphologically transformed phenotype appeared to revert.

### Example 10

### Induction of Proliferation of Human Primary Osteoblast Cells by the 30664188 Protein

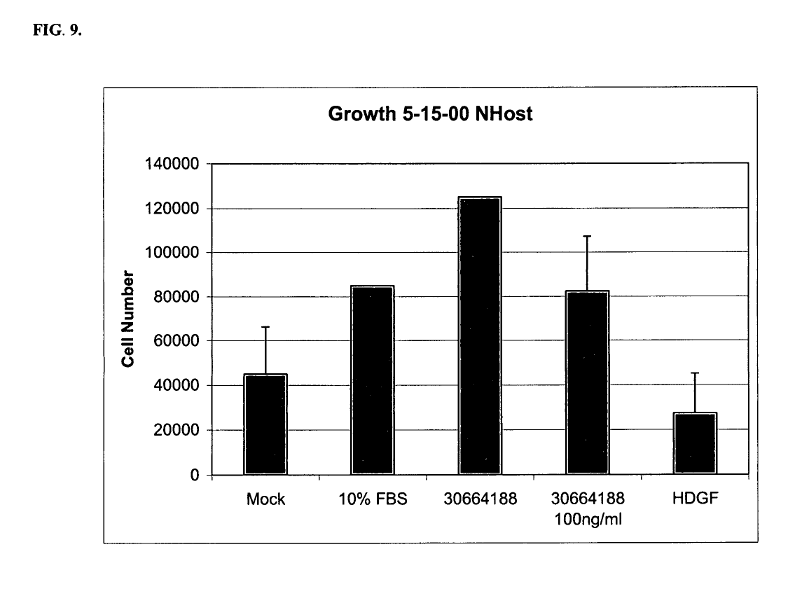

In an experiment similar to that described in Example 9, human primary osteoblast cells (NHost; Clonetics) also underwent a dose-dependent increase in cell number by 3- to 4-fold (FIG. 9). The dose required to elicit a 50% response in FIG. 9 is below 100 ng/mL of pCEP4/Sec/30664188.m99.

In summary, the observations that the 30664188 protein induces DNA synthesis (Example 8), cell growth (Examples 9 and 10), and morphological transformation (Example 9) indicate that the protein possesses transforming properties.

### Example 11

### Induction of Tumor Formation by the 30664188 Protein

NIH 3T3 cells with treated conditioned medium from cells transfected with pCEP4/Sec or pCEP4/Sec/30664188 were cultured as described above. 10⁶ cells in 0.1 mL PBS were then injected subcutaneously into the lateral subcutis of female nude mice (Charles River Laboratory), n=5 per group (termed, e.g., pCEP4/Sec/30664188.m99 mice). After 11 and 14 days, tumor formation was assayed with calipers.

After 11 days, tumor growth was evident in pCEP4/Sec/30664188.m99 mice. pCEP4/Sec/30664188.m99 mice (5/5) were positive for tumor formation with tumor size measuring 6.74±0.58 mm3. After 14 days in culture a noticeable decrease in tumor size was evident in pCEP4/Sec/30664188.m99 mice with 3/5 mice positive and average tumor volume 1.44±0.88 mm3. Notably, and as a positive control, 5 of 5 mice treated with bFGF developed tumors which increased in volume to 66.56±13.2 mm3. Control vector mice (0/5) were negative for tumor formation. Although these data strongly suggest that 30664188.m99 overexpression induces tumor formation in nude mice, tumors appeared to be lost as a function of time. Strikingly, these data parallel the morphological reversion properties noted in the NIH 3T3 transformation assay.

### Example 12

### Purification of Intact and Cleaved Products of the 30664188.m99 Protein.

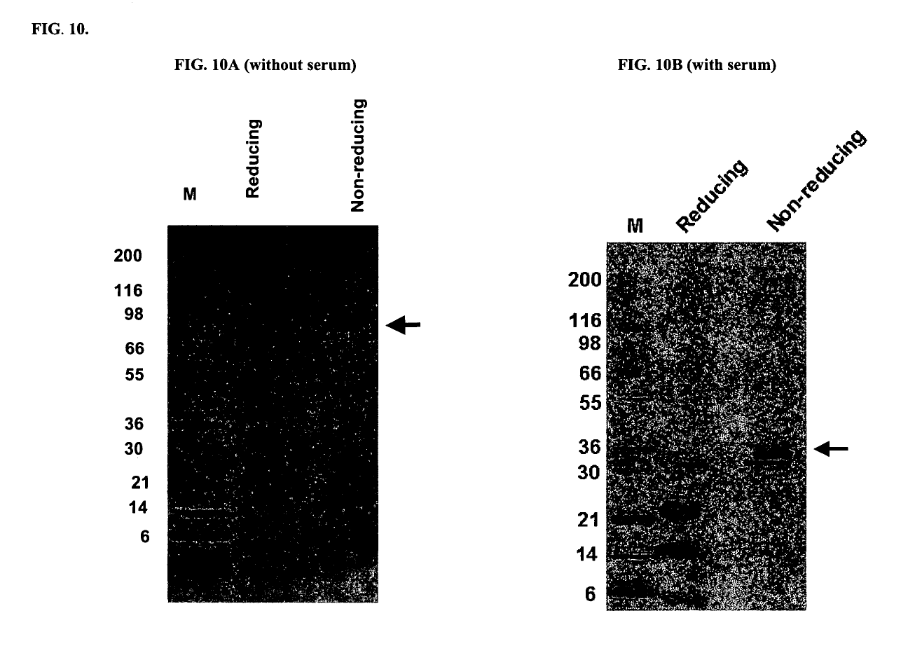

It was observed that in certain experiments treatment with the vector pCEP4/Sec/30664188.m99 did not result in DNA synthesis or cell proliferation. In additional experiments, medium conditioned with 30664188.m99 was obtained from HEK 293 cells grown in the presence of serum (Example 6). The 30664188.m99 gene product was purified by cation exchange chromatography, followed by nickel affinity chromatography. The protein product was run under nonreducing and reducing conditions on SDS-PAGE, and developed by Coomassie stain. The results are shown in FIGS. 10A and 10B. In the presence of serum, the 30664188.m99 gene product appeared as a protein of about 35 kDa under nonreducing conditions (FIG. 10B. However, this polypeptide appears as three degraded bands when run under reducing conditions. The apparent molecular weights of the two bands were 22-25 kDa (band I), about 16 kDa (band II) and about 5-6 kDa (band III). N-terminal amino acid analysis of these fragments indicates that bands I and II both begin at residue 247 of the 30664188.m99 amino acid sequence, and that band III begins at residue 339. These results are consistent with cleavage of the polypeptide corresponding to band I to provide the fragments of bands II and III. It is possible that the 35 kDa band observed under nonreducing conditions is a dimer composed of band I, and/or the bonded polypeptide composed of bands II and III, observed under reducing conditions.

Amino terminal analysis indicates that the gene product from pCEP4sec/30664188.m99-transfected 293 cells grown in the presence of serum, isolated according to the procedure described above, is a carboxyl-terminal fragment of the full length protein. The 35 kDa band found under nonreducing conditions is termed p35 below.

When 293 cells were cultured in the absence of serum, and the same isolation and detection procedure described in the preceding paragraph is followed, a different gene product is observed. Under nonreducing conditions a band was found at about 85 kDa (FIG. 10A). This protein is termed p85 below. The corresponding gene product observed under reducing conditions a major band is found at about 53-54 kDa. N-terminal amino acid analysis of this gene product provides the amino acids at the multiple cloning site used in pCEP4sec/30664188.m99 (Example 6). The residues corresponding to the Ig kappa leader sequence, cloned upstream from the multiple cloning site, are absent. These results indicate that the gene product obtained in the absence of serum represents the full amino acid sequence encoded in pCEP4sec/30664188.m99. The p85 polypeptide is thought to be a dimer of the 50 kDa species observed on reducing SDS-PAGE.

### Example 13

### Activity of Intact and Cleaved Fragments of the 30664188.m99 Protein

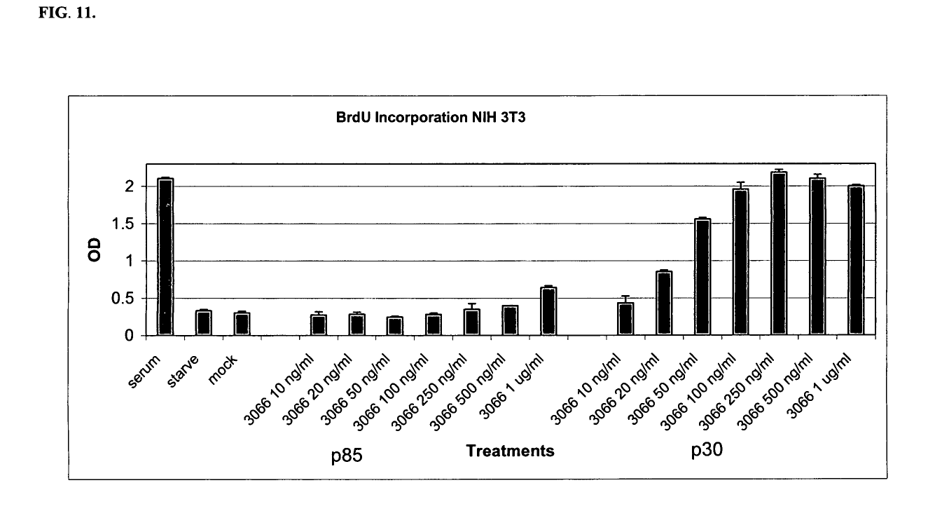

Purified p85 and p35 FCTRX proteins were separately applied to NIH 3T3 cells in a range of concentrations. Incorporation of BrdU was evaluated as described in Example 8. The results are shown in FIG. 11. It is seen that p85 has growth-promoting activity that does not differ from control levels except at the highest concentration used. p35, on the other hand, was at least as active, if not more so, than unfractionated pCEP4/Sec/30664188 conditioned medium. The concentration of p35 giving 50% of the maximum DNA synthesis falls between 20 and 50 ng/mL.

These results suggest that the p35 fragment derived from intact 30664188.m99 has growth-promoting activity but that the intact dimeric form of the .m99 protein, p85, does not. Therefore, reversion of transformation and tumor formation seen in Examples 9 and 11 may be the result of the emergence of a species in the culture at such longer times that inhibits or prevents formation of a p35-like species from p85.

### Example 14

### Phosphorylation of the PDGF Alpha Receptor

Binding of the p35 fragment and the p85 dimer forms of 30664188.m99 protein to platelet-derived growth factor (PDGF) alpha and beta receptors was examined.

Binding was measured by assaying receptor tyrosine phosphorylation. Tyrosine residues of the alpha PDGF receptor become phosphorylated as a result of p35 treatment to an extent greater than that elicited by the binding of an equivalent amount of PDGF AA, but to a slightly lesser extent than that induced by PDGF BB. The p35-induced BrdU incorporation in NIH 3T3 cells and transformation of the cells was inhibited by treatment with a polyclonal antibody specific for the PDGF alpha receptor.

These results demonstrate that the p35 fragment induces activation of the alpha PDGF receptor. The p35-induced activation was not inhibited as completely as that of PDGF AA, suggesting that activation of an additional signaling mechanism occurs.

Analysis of activation of the PDGF beta receptor demonstrated that p35 and PDGF BB induce beta PDGF receptor tyrosine phosphorylation to a similar extent. Treatment with p85 did not activate the beta PDGF receptor. The PDGF beta receptor may mediate the additional signaling noted above in the experiments with the PDGF alpha receptor.

These experiments show that inhibition or abrogation of the interaction of p35 with an important receptor involved in its signaling pathway prevents molecular events from occurring that are most likely implicated in its signaling mechanism.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising an isolated polypeptide comprising amino acids 24-370 of SEQ ID NO:2 or the corresponding amino acids of SEQ ID NO:4.

2. The composition of claim 1 further comprising a pharmaceutically acceptable carrier.

3. The composition of claim 1, wherein the polypeptide has growth factor activity.

4. The composition of claim 3, wherein the growth factor activity is a platelet-derived growth factor activity.

5. The composition of claim 3, wherein the growth factor activity is involved in an inflammatory pathology in a subject.

6. The composition of claim 5 wherein the subject is a mammal.

7. The composition of claim 5 wherein the subject is a human.

8. The composition of claim 5 wherein the inflammatory pathology is inflammatory bowel disease.

9. The composition of claim 5 wherein the inflammatory pathology is nephritis.

10. The composition of claim 5 wherein the inflammatory pathology is Crohn's disease.

11. The composition of claim 2 wherein the composition is suitable for intravenous administration to a subject.

12. The composition of claim 2 wherein the composition is suitable for subcutaneous administration to a subject.

13. A composition comprising a polypeptide dimer comprising two associated polypeptides, wherein each polypeptide comprises amino acids 24-370 of SEQ ID NO:2 or the corresponding amino acids of SEQ ID NO:4.

14. The composition of claim 13 further comprising a pharmaceutically acceptable carrier.

15. The composition of claim 13, wherein at least one of the polypeptides is proteolytically cleaved at Arg247 of SEQ ID NO:2.

16. The composition of claim 1, wherein the polypeptide comprises an epitope.

17. The composition of claim 16, wherein the epitope comprises at least 6 amino acid residues, at least 10 amino acid residues, at least 15 amino acid residues, at least 20 amino acid residues, or at least 30 amino acid residues.

18. The composition of claim 16, wherein the epitope forms a specific immune complex with an antibody raised against the polypeptide.

19. The composition of claim 16, wherein the epitope is located in a hydrophilic region of the polypeptide.

20. The composition of claim 19, wherein the hydrophilic region of the polypeptide is on the surface of the polypeptide.


SEQUENCE LISTING (24 sequences)

SEQ ID NO: 1 - DNA, 1828 bp, Homo sapiens
  CDS (182)..(1291)
ctaaaaaatatgttctctacaacaccaaggctcattaaaatattttaaatattaatatacatttcttctgtcagaaatacataaaactttattatatcagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgagagcaatcacctcacagacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgtgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttg

SEQ ID NO: 2 - protein, 370 aa, Homo sapiens
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 3 - DNA, 1587 bp, Homo sapiens
  CDS (540)..(935)
agaggctctcaaattagatcaagaaatgcctttaacagaagtgaagagtgaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgtgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaatatggtttccagagaaaaggccaatacctaagcattttttccatgagaagcactgcatacttacctatgtggactataataacctgtctccaaaac

SEQ ID NO: 4 - protein, 132 aa, Homo sapiens
MYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 5 - DNA, 38 bp, Artificial Sequence
  chemically synthesized sequence
ctcgtcgaattcaccccgcagagcgcatccatcaaagc

SEQ ID NO: 6 - DNA, 39 bp, Artificial Sequence
  chemically synthesized sequence
ctcgtcctcgagtcgaggtggtcttgagctgcagataca

SEQ ID NO: 7 - DNA, 21 bp, Artificial Sequence
  chemically synthesized sequence
ggacgatggtgtggacacaag

SEQ ID NO: 8 - DNA, 21 bp, Artificial Sequence
  chemically synthesized sequence
cttgtgtccacaccatcgtcc

SEQ ID NO: 9 - DNA, 21 bp, Artificial Sequence
  chemically synthesized sequence
tatcgaggcaggtcataccat

SEQ ID NO: 10 - DNA, 21 bp, Artificial Sequence
  chemically synthesized sequence
atggtatgacctgcctcgata

SEQ ID NO: 11 - DNA, 30 bp, Artificial Sequence
  chemically synthesized sequence
ctcgtcctcgagggtaagcctatccctaac

SEQ ID NO: 12 - DNA, 31 bp, Artificial Sequence
  chemically synthesized sequence
ctcgtcgggcccctgatcagcgggtttaaac

SEQ ID NO: 13 - DNA, 14 bp, Artificial Sequence
  chemically synthesized sequence
catggtcagcctac

SEQ ID NO: 14 - DNA, 14 bp, Artificial Sequence
  chemically synthesized sequence
tcgagtaggctgac

SEQ ID NO: 15 - DNA, 20 bp, Artificial Sequence
  chemically synthesized sequence
cgcttggcatcatcattgag

SEQ ID NO: 16 - DNA, 21 bp, Artificial Sequence
  chemically synthesized sequence
cggtatcgaggcaggtcatac

SEQ ID NO: 17 - DNA, 27 bp, Artificial Sequence
  chemically synthesized sequence
tccaggtcaacttttgacttccggtca

SEQ ID NO: 18 - DNA, 24 bp, Artificial Sequence
  chemically synthesized sequence
cacaaggaagttcctccaaggata

SEQ ID NO: 19 - DNA, 26 bp, Artificial Sequence
  chemically synthesized sequence
aatccaggtttagccacaaagtagtc

SEQ ID NO: 20 - DNA, 35 bp, Artificial Sequence
  chemically synthesized sequence
agaacgaaccaaattaaaatcacattcaagtccga

SEQ ID NO: 21 - DNA, 20 bp, Artificial Sequence
  chemically synthesized sequence
gcatgtgcaggacctccagt

SEQ ID NO: 22 - DNA, 18 bp, Artificial Sequence
  chemically synthesized sequence
tccacgttgcctcctcgt

SEQ ID NO: 23 - DNA, 27 bp, Artificial Sequence
  chemically synthesized sequence
cagttccacagccacaatttcctccac

SEQ ID NO: 24 - protein, 345 aa, Homo sapiens
MSLFGLLLLTSALAGQRQGTQAESNLSSKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG